(19) United States
(12) Patent Application Publication
(10) Pub. No.: US 2024/0165518 A1
(43) Pub. Date: May 23, 2024

(54) METHODS FOR ADAPTIVE BEHAVIORAL TRAINING USING GAZE-CONTINGENT EYE TRACKING AND DEVICES THEREOF

(71) Applicant: BIOSTREAM TECHNOLOGIES, LLC, Moorestown, NJ (US)

(72) Inventors: Benjamin Farber, Boston, MA (US); Sidney Luc Robinson, Toronto (CA); Michael Farber, Moorestown, NJ (US)

(21) Appl. No.: 18/557,845

(22) PCT Filed: Apr. 28, 2022
(86) PCT No.: PCT/US2022/026773

§ 371 Date: Oct. 27, 2023

Related U.S. Application Data
(60) Provisional application No. 63/180,748, filed on Apr. 28, 2021.

(51) Int. Cl. A63F 13/67; G06F 3/01; G06V 40/16; G16H 20/70

(52) U.S. Cl. CPC A63F 13/67; G06F 3/013; G06V 40/174; G06F 2203/011; G16H 20/70

(57) ABSTRACT

The technology discloses providing a three-dimensional gameplay via a graphical user interface to a user device, wherein the provided three-dimensional gameplay prompts a response from a user device and the response is tracked via a gaze contingent technique. Next, performance data is identified based on the received response from the user device. A difficulty level of the provided three-dimensional gameplay is adjusted based on the identified performance data. The adjusted three-dimensional gameplay is provided to the user device to track additional performance data.

This application claims the benefit of U.S. Provisional Patent Application Ser. No. 63/180,748, filed Apr. 28, 2021, which is hereby incorporated by reference in its entirety.

### TECHNICAL FIELD

The present application relates generally to devices, systems, processes, and methods for performing adaptive behavioral training using gaze-contingent eye tracking and devices thereof.

### BACKGROUND

Autism spectrum disorder (ASD) is characterized by deficits in social communication and interaction, as well as restricted repetitive patterns of behavior, interests, or activities. Children with ASD display deficits in emotion recognition (ER), which is the ability to identify emotions in themselves and others. Emotion recognition is a crucial part of social development and viewed as a basic ability that underlies more complex emotional understanding and social skills. Facial emotion recognition (FER) may be utilized in analyzing the environmental cues related to emotional behavior, and eye tracking studies have consistently supported deficits in FER in youth with ASD across various emotional expressions (e.g., anger, sadness, happiness, fear). The ability to visually scan the face in its entirety is imperative to teaching discriminations of facial cues within an emotional context. Individuals with ASD tend to focus on less emotionally expressive or relevant portions of the face, such as greater emphasis on scanning of the mouth rather than the eyes or upper portions of the face. This has important implications for ER as increased gaze directed at the eyes or decreased gaze at areas outside the mouth and eyes has been related to better ER among youth with ASD.

Effective behavioral interventions exist for the remediation of social skill deficits in children with ASD. Behavior analytic therapeutic approaches for improving social skills and ER, such as discrete trial training (DTT), video modeling, and peer-mediated instruction are considered effective interventions. Naturalistic Developmental Behavioral Interventions (NDBIs) that utilize naturalistic play environments to deliver interventions combining behavioral and developmental principles have also been demonstrated to be effective and offer advantages for the generalizability of some skills. The main instructional challenge with these approaches is the need for intensive teaching and for vast numbers of learning opportunities. This requires significant time from trained professionals with specialized expertise who are often not accessible due to a shortage of supply. Thus, it is imperative to identify approaches that serve as adjunctive therapies to improve ER Specifically, approaches that can be administered semi-autonomously, with less time and supervision required, resulting in behavioral improvements and resource advantages.

Video game platforms as a digital therapeutic help overcome these barriers and are gaining popularity as an effective and empirically-supported means of augmenting behavioral interventions for children with ASD and other neurodevelopmental disorders. The Food and Drug Administration (FDA) recently cleared the first prescription-only game-based digital device for treatment of attention deficit hyperactivity disorder, a neurodevelopmental condition. Computer games targeting improvement in facial recognition using didactic instructions and lessons, imitation exercises, repeated practices, and quizzes or games matching emotional depictions have resulted in improvements in ER, facial recognition, and social interactions. The structured repetition, discreteness, and focus on specific components of complex skills in DTT approaches may make these types of behavioral interventions particularly suitable for delivery via gaming platforms.

However, these gaming platforms have not largely implemented gaze-contingent eye tracking (GCET) which would allow for immediate reinforcement of successive approximations toward the terminal behaviors (e.g., targeted visual behaviors including facial scanning). Without this specific and precise gaze data, an intervention cannot measure or reinforce these gaze behaviors that are necessary for building ER skills. One training program that used GCET to trigger events designed to increase attention to faces presented in a video produced encouraging findings among 3-year-old children with ASD. Use of GCET embedded in a gaming platform may create a potent reinforcement mechanism for desired gaze behaviors in this population given nearly half of youth with ASD choose to use electronic or computer games during their free time. This reinforcement mechanism also allows pairing a non-social reinforcer (video games) with socially mediated instruction, which may establish social learning as a reinforcing activity.

In addition, the widespread use and scientific acceptance of eye tracking technology, the development of a new generation of lightweight, compact and wireless physiological monitoring devices (including, without limitation, electroencephalogram (“EEG”), electrocardiogram (“ECG”), or galvanic skin resistance measuring device (“GSR”)), software to capture and synchronize the data collected from these devices, and advances in cloud based machine learning and artificial intelligence systems, has provided the opportunity for creation of a device or system for behavioral training (including visual training) of individuals while also training the user to reach and/or maintain targeted mental, emotional, physiological and behavioral states when engaged in training activities (including while engaged in simulation-based training) based on many different parameters. Individuals with certain medical conditions, including autism spectrum disorder, can benefit from such a highly personalized training system that applies the optimal combination of parameter values to achieve maximum benefits over time as the individual's proficiency increases.

Similarly, individuals who must perform potentially life-saving functions under extremely stressful conditions (such as medical and police first-responders and other emergency personnel) where maintaining mental focus and a calm emotional state, while performing some form of visual analysis represents an essential part of achieving successful outcomes, as well as others who must engage in visual analysis while maintaining mental focus under stressful conditions (such as athletes under the stress of extreme competition) could also benefit from the training provided by this device or system The device or system also functions as an assessment and/or diagnostic tool by enabling the establishment of correlations between user data and the presence of certain medical and neurological conditions of users.

### SUMMARY

The technology discloses a method including, providing a three-dimensional gameplay via a graphical user interface to a user device, wherein the provided three-dimensional gameplay prompts a response from a user device and the response is tracked via a gaze contingent technique. Next, performance data is identified based on the received response from the user device. A difficulty level of the provided three-dimensional gameplay is adjusted based on the identified performance data. The adjusted three-dimensional gameplay is provided to the user device to track additional performance data.

In another embodiment, the identified performance data and the additional performance data is stored at a server. The stored performance data and the additional performance data associated with a user of the user device is analyzed. One or more therapeutic progress reports based on the analysis is generated.

In another embodiment, the provided three-dimensional gameplay comprises simulated social interactions with three-dimensional animated characters.

In yet another embodiment, the response comprises data from an eye region, and a mouth region of the user using the user device tracked via the gaze contingent technique.

In another embodiment, adjusting the three-dimensional gameplay minimizes user fatigue and maintains a user engagement time.

In yet another embodiment, the adjusting further comprises, adjusting the difficulty level of the provided three-dimensional gameplay based on a feedback data received from a therapy or education provider.

Additional features and advantages of this disclosure will be made apparent from the following detailed description of illustrative embodiments that proceeds with reference to the accompanying drawings.

### BRIEF DESCRIPTION OF THE DRAWINGS

The foregoing and other aspects of the present invention are best understood from the following detailed description when read in connection with the accompanying drawings.

For the purpose of illustrating the invention, there are shown in the drawings embodiments that are presently preferred, it being understood, however, that the invention is not limited to the specific instrumentalities disclosed. Included in the drawings are the following Figures:

FIG. 1 provides an illustrative example of component interaction and data flow, according to some embodiments of the present invention;

FIG. 2A shows an example of a visual training area (VTA) displayed in a visual presentation, according to some embodiments;

FIG. 2B shows a first example of how the VTA shown in FIG. 2A can be narrowed based on measurement data collected from a user, according to some embodiments;

FIG. 2C shows a second example of how the VTA shown in FIG. 2A can be narrowed based on measurement data collected from a user, according to some embodiments;

FIG. 2D shows an example of how the VTA shown in FIG. 2A can be presented without a visual prompt, according to some embodiments;

FIG. 3A shows an example of a VTA displayed in a visual presentation with two human faces, according to some embodiments;

FIG. 3B shows an example of how the VTA depicted in FIG. 3A can be moved to a different area of the visual presentation based on measurement data collected from a user, according to some embodiments;

FIG. 3C shows an additional example of how the VTA depicted in FIG. 3A can be moved to a different area of the visual presentation based on measurement data collected from a user, according to some embodiments;

FIG. 4A shows an example of a VTA displayed in a visual presentation, according to some embodiments;

FIG. 4B shows a first example of how the shape of the VTA shown in FIG. 4A can be morphed based on measurement data collected from a user, according to some embodiments;

FIG. 4C shows a second example of how the shape of VTA shown in FIG. 4A can be morphed based on measurement data collected from a user, according to some embodiments;

FIG. 5 shows an example of presenting two VTAs in a single visual presentation, according to some embodiments;

FIG. 6 shows a second example of presenting two VTAs in a single visual presentation, according to some embodiments;

FIG. 7A presents an example of a first step of simulated joint attention exercise where a graphical depiction of a car and a human face are presented in visual presentation along with a VTA defined around the eyes of the human face, according to some embodiments;

FIG. 7B presents a second step of the simulated joint attention exercise shown in FIG. 7A where the visual presentation is updated;

FIG. 7C presents a third step of the simulated joint attention exercise shown in FIG. 7A where the VTA is moved from the human face to the car;

FIG. 7D presents a fourth step of the simulated joint attention exercise shown in FIG. 7A where the VTA is moved from the car back to the face;

FIG. 8 presents examples of how the visual presentation may be modified in response to movement of the user, according to some embodiments;

FIG. 9 presents additional examples of how the visual presentation may be modified in response to movement of the user, according to some embodiments;

FIG. 10A illustrates the first step of a process to train individuals to recognize the emotions of others using VTAs that are determined by both eye tracking measurement data and behavioral measurement data, according to some embodiments;

FIG. 10B illustrates the second step of a process to train individuals to recognize the emotions of others using VTAs that are determined by both eye tracking measurement data and behavioral measurement data, according to some embodiments;

FIG. 10C illustrates the third step of a process to train individuals to recognize the emotions of others using VTAs that are determined by both eye tracking measurement data and behavioral measurement data, according to some embodiments;

FIG. 10D illustrates the fourth step of a process to train individuals to recognize the emotions of others using VTAs that are determined by both eye tracking measurement data and behavioral measurement data, according to some embodiments;

FIG. 11A illustrates an example of training individuals to make and/or maintain eye contact in real world interactions based on eye tracking data collected during a visual presentation in which physiological and/or behavioral measurement data, according to some embodiments.

FIG. 11B shows an alternative view of the example presented in FIG. 11A;

FIG. 12A shows an example of a training process where feedback collected using a physiological measuring device is used to updated the visual presentation, according to some embodiments;

FIG. 12B shows the example of FIG. 12A with visual prompts to direct the user to VTAs, as may be implemented in some embodiments;

FIG. 13A shows an example of a training process where a user is presented with a list of possible actions in text format, according to some embodiments;

FIG. 13B illustrates how a prompt for a VTA may be added to the example of FIG. 13A;

FIG. 13C illustrates how a second prompt for a VTA may be added to the example of FIG. 13B;

FIG. 14A illustrates how visual presentations, according to the techniques described herein, can be used to train emergency medical personnel as part of training simulations;

FIG. 14B provides a second example of how visual presentations, according to the techniques described herein, can be used to train emergency medical personnel as part of training simulations;

FIG. 15A illustrates how visual presentations, according to the techniques described herein, can be used to train forensic law enforcement personnel as part of training simulation;

FIG. 15B provides a second example of how visual presentations, according to the techniques described herein, can be used to train forensic law enforcement personnel as part of training simulation;

FIG. 16 illustrates an example interface that may be used by service provider for entering data into the system described herein;

FIG. 17 illustrates a computer-implemented method for adaptive behavioral training, according to some embodiments; and

FIG. 18-21 illustrates flow diagrams and tables of an exemplary embodiment.

### DETAILED DESCRIPTION OF EXEMPLARY EMBODIMENTS

The following disclosure describes the present invention according to several embodiments directed at methods, systems, and apparatuses related to performing adaptive behavioral training, and training of associated physiological responses, with assessment and diagnostic functionality. In particular, the techniques described herein utilize visual training areas or “VTAs” in visual presentations. The term “VTA” refers to an area of the visual presentation, where the visual presentation may be defined by a set of coordinates, and the VTA may be defined by a set of coordinates from the set of coordinates that define the visual presentation. The VTA may overlay a single or multiple visual representations of anything presented in the visual presentation included but not limited to persons, places, and/or things and/or a region or regions thereof. Examples of the set of coordinates defining the VTA include coordinates that create an oval shaped VTA for eye contact exercises; coordinates encompassing the entire visual presentation field in the case of the head positioning example; and coordinates that create more than one overlay over different faces within the visual presentation. In some embodiments, the VTA is visible to the user within the visual presentation, while in other embodiments, the VTA is not visible. Examples of visual presentations in which VTAs may be presented include, without limitation, video games, virtual reality generated experiences, real world presentations in which eye tracking glasses are used and augmented reality presentations. Following presentation of a VTA to a user, measurement data is collected indicating how the user is reacting to the presentation of the VTA Then, based on this measurement data, the VTA may be modified or other training procedures may be performed.

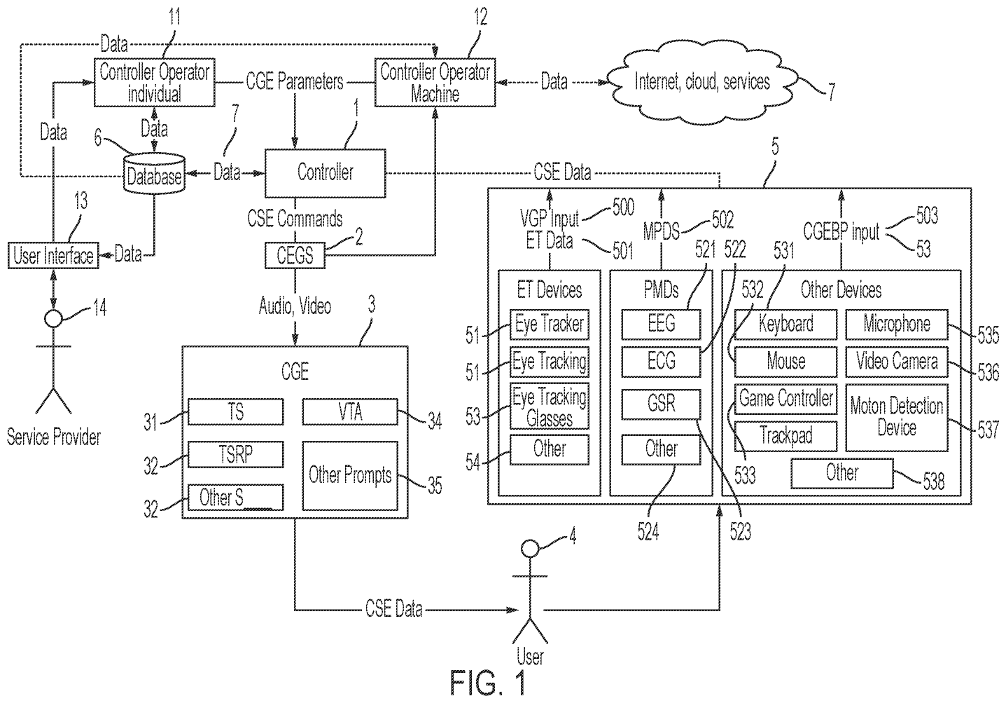

FIG. 1 provides an illustrative example of component interaction and data flow, according to some embodiments of the present invention. In this example, an Eye Tracker (ET) device 51 coupled with software that provides for transmission of eye tracking data (“ET Data”) to the Controller 1. ET devices are known in the art and generally any ET device may be used with the technology described herein.

A Computer Experience Generation System (“CEGS”) is used. The CEGS is a system (which could include combinations of different software and hardware) that generates a Computer Generated Experience (“CGE”). The CGE is an interactive graphical user interface (“GUI”) that may include, for example, text, images, animations, videos, audio, touch sensory experiences, a video game, use of computer based devices including robots, etc. or any combination thereof and which includes a form of visual presentation to the user. It should be noted that, although the CGE includes a visual presentation, the CGE does not generate the visual presentation. For example, where the CGE is integrated with real world eye tracking glasses, augmented reality techniques may be employed where the user views a real world object and is presented with a VTA within a region of the real world object.

The visual presentation may include an electronically generated visual presentation or a real world visual presentation, or any combination thereof. Each visual presentation may be defined in a coordinate space specified, for example, based on the operating environment of the visual presentation. For example, for electronically generated visual presentation, the coordinate space may be a Cartesian coordinate space bounded by the dimensions of the screen or window in which the visual presentation is displayed. In general, any coordinate space known in the art may be used for displaying the visual presentation.

The CEGS may include different components including but not limited to a computer, computer monitor, mobile computing device such as a smartphone, television, computer software for creation and presentation of CGEs, computer software for collection and transmission of the user's behavioral and/or physiological data while engaged in a CGE, audio devices including speakers and headphones, virtual reality devices (such as virtual reality headset), real world eye tracking glasses, devices and/or systems that generate an augmented reality experience so that the CGE is presented to the user as a visual overly to real world visual experiences, and devices and/or systems that can create touch sensory experiences, and any combination of these components. The CEGS can receive instructions in the form of CGE Commands from the Controller 1 and alter the CGE based on those instructions.

As shown in the example of FIG. 1, the CGE 3 includes a VTA 34 which is an area of the visual presentation that is defined by a set of coordinates which may be from the set of coordinates that define the visual presentation. The VTA 34 may overlay a single or multiple visual representations of anything presented in the visual presentation included but not limited to persons, places, and/or things and/or a region or regions thereof. The VTA 34 may or may not be visible to the user within the visual presentation and may include visual indicator of the VTA 34 including through a graphical representation of the boundary of the VTA 34. VTAs may take different forms (including but not limited to in different sizes, geometric shapes, and locations), and be presented to the user concurrently or presented sequentially at different times and locations (which may or may not be graphically designated), as part of the visual presentation upon which the user is to focus visual attention for at least one segment of time during the CGE 3. Eye tracking measurement data indicating the user's gaze with respect to the VTA 34 is collected (such user's eye tracking measurement data is hereinafter referred to as “Visual Gaze Performance Input”). VTAs may be presented in different patterns, different forms (including but not limited to in different sizes, geometric shapes, and locations), and may be presented to the user concurrently or presented sequentially at different times and locations which may be determined by CGE Commands and based on CGE Parameters.

The system, including as shown in FIG. 1, may provide for the CGE 3 to include an interactive experience (including Training Stimulus, Training Stimulus Response Prompt, and Training Behavioral Response Input, as described below) where the user provides an input and/or any combination of different inputs at a single point in time or at varying points in time during the CGE 3 (including but not limited to through use of a video game controller, motion controller devices and/or systems such as a Nintendo Wii, Sony PlayStation Move, and Microsoft Kinect and other devices that incorporate use of an accelerometer to capture motion data, webcam for inputting of certain physical movements of the user including facial expression, microphone for inputting of speech and other vocalization by the user, touchscreen, mouse, keyboard, virtual reality headset, etc.) excluding Visual Gaze Performance Input and ET Data, which inputs shall hereinafter be referred to as “CGE Behavioral Performance Input”.

During the CGE the user may be presented with a stimulus or stimuli (in the form of a single or combination of visual (including a VTA), auditory, and/or other sensory stimulus) designed to train the user's mental, emotional, physiological and/or behavioral response to such stimulus or stimuli (“Training Stimulus”).

Prior to, during, or following presentation of the Training Stimulus, the user may be prompted by the CGE to take and/or decide on a specific action or combination of actions in response to the Training Stimulus (including but not limited to choosing an action or combination of actions from a group of possible actions presented during the CGE and/or creating an action or combination of actions in response to the Training Stimulus) (“Training Stimulus Response Prompt”). As an example, a Training Stimulus Response Prompt in the form of a graphical representation of the boundaries of a VTA is presented to the user. In some embodiments, a dotted line may be used to designate the boundaries of the VTA. In other embodiments, other representations may be used (e.g., shading or blurring of regions outside of the boundaries). As a second example, an auditory prompt (including in the form of a sound or verbal instruction) may be used to prompt the user to direct the user's gaze to the VTA.

In some embodiments, the system may also provide the user with the ability to provide a CGE Behavioral Performance Input and/or Visual Gaze Performance Input in response to the Training Stimulus Response Prompt (“Training Behavioral Response Input”).

In some embodiments, the system provides for the transmission, recording and storage of all data with respect to the stimuli presented to the user by the system (which could include timing and nature of certain visual stimuli presented to the user in descriptive and numeric text format and in video screen recordings) and the user's responses to the stimuli (collectively referred to as “CGE Data”) via communication linkage between the Eye Tracker 511, CEGS 2, the Controller 1, and the Database 6, via a combination of communication methods such as a direct USB connection, an Application Programming Interface, and executable software routines and protocols. CGE Data may include, for example, the ET Data, VTAs presented to the user (“VTA Data”), Training Stimulus and Training Stimulus Response Prompts presented to the user (“Training Stimulus Data”), the user's Visual Gaze Performance Input (“Visual Gaze Performance Input Data”), the user's CGE Behavioral Performance Input (“CGE Behavioral Performance Input Data”) and all data with respect to the Training Behavioral Response Input (“Training Behavioral Response Input Data”).

The system, including as shown in the example in FIG. 1, may also include a Computer Database used and configured to receive and store the CGE Data (including ET Data, VTA Data, Visual Gaze Performance Input Data, CGE Behavioral Performance Input Data, and Training Behavioral Response Input Data), CGE Commands, and CGE Parameters, that can transmit to and receive data from the Controller.

The system includes a Controller Operator, which is an individual and/or machine that inputs and/or transmits CGE Parameters to the Controller 1. In the example of FIG. 1, the Controller Operator includes Service Provider 14 and possibly machine-generated data received over Internet cloud services 7 and/or via the CEGS 2 (as described in further detail below). Software at the Controller 1 receives CGE Data in real time and based on CGE Data and parameters defined by the Controller Operator, generates instructions to alter the CGE including the Training Stimulus and Training Stimulus Response Prompts (“CGE Commands”), and transmits these CGE Commands to the CEGS to alter the CGE including the Training Stimulus and Training Stimulus Response Prompts. The parameters defined by the Controller Operator (referred to herein as the “CGE Parameters”) may include, for example, fixed values, value ranges, and rules based on values and/or value ranges, and they may be generated by individuals and/or pre-programmed algorithms.

CGE Commands can include, for example, instructions (which can be applied in real time or in subsequent CGEs) with respect to the VTA including but not limited to user's required time to make initial visual contact with the VTA, required time to maintain continuous visual contact within the VTA, permissible time to stop and then resume visual contact with the VTA (deviation tolerance), shape of the VTA, size of the VTA, changing shape and/or size (including real time morphing) of the VTA while the user maintains visual contact within the VTA or at some later moment in time, change in position of the VTA in the CGE environment such as on the computer monitor or in the user's visual field in the real world environment (as in the case of an augmented reality application), degree of visual distraction occurring at or near the VTA and/or auditory distraction.

CGE Commands can also include instructions (which can be applied in real time or in subsequent CGEs) with respect to the CGE other than the VTA including changes in the type, nature, and timing of the CGE experienced by the user for other purposes including but not limited to changes in Training Stimulus and Training Stimulus Response Prompts for adaptation of training simulations and/or for the purpose of maintaining and optimizing engagement of the player during the CGE.

In some embodiments, CGE Parameters can use data related to the user's prior performance and/or behavioral data as associated with any VTA or a combination of VTAs including but not limited to the user's time to make initial visual contact with the VTA, time the user maintained continuous visual contact within the VTA, the user's deviation from contact with the VTA during the time required for continuous visual contact, shape of the VTA which the user experienced, size of the VTA which the user experienced, changes in shape and/or size (including real time morphing) of the VTA which the user experienced including while the user maintained visual contact within the VTA, changes in position of the VTA in the CGE environment which the user experienced such as changes in position of the VTA on a computer monitor or in the user's perceived visual field in a real world environment (as in the case of an augmented reality application) and degree of visual distraction experienced at or near the VTA and/or auditory distraction.

CGE Parameters may also include use of: (i) CGE Data related to the user's current and/or prior performance and/or behavior during a CGE (including but not limited to VTA Data, Visual Gaze Performance Input Data, CGE Behavioral Performance Input Data, Training Stimulus Data, and Training Behavioral Response Input Data), (ii) other data associated with the user excluding CGE Data (such as age, education, gender, and medical diagnosis), (iii) the CGE Data of other users, (iv) the data of other users excluding CGE Data, and (iv) the data of non-users of the system or any other available data or information (including any or all such data collected prior to the user's then current use of the system and/or collected concurrently with the user's then current use of the system).

The system, including as shown in the example in FIG. 1, may also provide for application of algorithms, including machine learning algorithms that internalize the CGE Data of the user, other data associated with the user aside from CGE Data, the CGE Data of other users, the data of other users excluding CGE Data, and the data of non-users of the system or any other available data or information, (including any or all such data collected prior to the user's then current use of the system and/or collected concurrently with the user's then current use of the system) to programmatically refine and/or create new CGE Parameters.

In some embodiments, the system is capable of generating customizable reports, including by providing an interface for system operators that provides for a communication link with the Database using one or more communication methods (such as an Application Programming Interface, and executable software routines and protocols) and includes the capability for system operators to create and apply simple and complex database queries to the Database to generate customized reports through such interface with respect to all CGE Data collected. Reports configured and/or generated can display training progress, diagnostic/assessment data or insights, and detailed reports describing associations or other insights within any subset of CGE Data collected (such as associations between Training Stimulus Data at any specific moment in time and the associated Training Behavioral Response Input Data and Visual Gaze Performance Input Data).

Continuing with reference to FIG. 1, according to another aspect of the present invention, the system may include a physiological measuring device (“PMD”) such as an EEG, ECG, GSR is used to collect data from a user during a CGE and is used to measure and transmit data with respect to a certain type of the user's physiological changes while engaging in a CGE (“Singular Physiologic Data Stream”) including such data associated with the user's response to Training Stimulus and/or to Training Stimulus Response Prompt (“Training Physiological Response Input”).

In some embodiments, The Singular Physiologic Data Stream is transmitted to the Controller in real time. Alternatively, or concurrently the Singular Physiologic Data Stream may be transmitted to the Computer Database in real time and stored in the Computer Database.

The CGE Data may include all data with respect to the Singular Physiologic Data Stream (“Singular Physiologic Data Stream Data”) including all data with respect to the Training Physiological Response Input (“Training Physiological Response Input Data”).

The user's current and/or prior Singular Physiologic Data Stream Data including Training Physiological Response Input Data can be incorporated into the CGE Parameters in real time (as captured) or in a future use of the system (as stored) including to deliver biofeedback like functionality to the user and/or create closed loop adaptation system functionality and/or improve performance by tailoring training activities to the user's physiologic state.

The current and/or prior Singular Physiologic Data Stream Data including the Training Physiological Response Input Data of other users can be incorporated into the CGE Parameters in real time (as captured) or in a future use of the system (as stored).

The system provides for application of algorithms, including machine learning algorithms that internalize the CGE Data of the user (including the user's current and/or prior Singular Physiologic Data Stream Data including Training Physiological Response Input Data), other data associated with the user excluding CGE Data, the CGE Data of other users (including the current and/or prior Singular Physiologic Data Stream Data including Training Physiological Response Input Data, of such other users), the data of other users excluding CGE Data, and the data of non-users of the system or any other available data or information, (including any or all such data collected prior to the user's then current use of the system and/or collected concurrently with the user's then current use of the system) to programmatically refine and/or create new CGE Parameters for deployment by the system. In general, any machine learning algorithm known in the art may be applied including, for example, algorithms based on artificial neural networks (“ANN”), deep learning, or learning classifier/regression systems.

In some embodiments, more than one PMD is placed on the user during a CGE and is used to concurrently measure and transmit data with respect to multiple types of the user's physiological changes while engaging in a CGE (“Multiple Physiologic Data Streams”) including such data associated with the user's response to Training Stimulus and/or to Training Stimulus Response Prompt.

Software may be used to synchronize the Multiple Physiologic Data Streams (“PMD Synchronization Software”) which may be included in the Controller. PMD synchronization software which may be included in the Controller can also be used to synchronize other CGE Data, including ET Data, VTA Data, Training Stimulus Data, Visual Gaze Performance Input Data, CGE Behavioral Performance Input Data, and Training Behavioral Response Input Data. In some embodiments, the PMD Synchronization Software is used to transmit the Multiple Physiologic Data Streams to the Controller in real time. In other embodiments, the PMD Synchronization Software is used to transmit the Multiple Physiologic Data Streams to the Database in real time and stored in the Database. In other embodiments, the PMD Synchronization Software is used to concurrently transmit the Multiple Physiologic Data Streams to both the Database and to the Controller in real time.

The CGE Data may include all data with respect to the Multiple Physiologic Data Streams (“Multiple Physiologic Data Streams Data”) including all data with respect to the Training Physiological Response Input (Multiple Data Streams). The user's current and/or prior Multiple Physiologic Data Streams Data including Training Physiological Response Input (Multiple Data Streams) Data can be incorporated into the CGE Parameters in real time (as captured) or in a future use of the system (as stored) including to deliver biofeedback like functionality to the user and/or create closed loop adaptation system functionality. The current and/or prior Multiple Physiologic Data Streams Data including the Training Physiological Response Input (Multiple Data Streams) Data of other users can be incorporated into the CGE Parameters in real time (as captured) or in a future use of the system (as stored).

In some embodiments, the system may be capable of generating customizable reports, including by providing an interface for system operators that provides for a communication link with the Database using one or more communication methods (such as an Application Programming Interface, and executable software routines and protocols) and includes the capability for system operators to create and apply simple and complex database queries to the Database to generate customized reports through such interface with respect to all CGE Data collected (including the user's Training Physiological Response Input (Multiple Data Streams) Data). Reports configured and/or generated can display training progress, diagnostic/assessment data or insights, and detailed reports describing associations or other insights within any subset of CGE Data collected (such as associations between Training Stimulus Data at any specific moment in time and the associated Training Behavioral Response Input Data and Training Physiological Response Input (Multiple Data Streams) Data).

The Service Provider from time to time may input and/or transmit CGE Parameters to the Controller with respect to the user based in whole or in part on the Service Provider's review and/or analysis of CGE Data collected with respect to the user including Training Stimulus Data and the associated Training Behavioral Response Input Data (which may be in the form of reports generated by the Service Provider's use of the system).

The Service Provider from time to time may also input and/or transmit CGE Parameters to the Controller with respect to the user based in whole or in part on the Service Provider's review and/or analysis of recommended CGE Parameters generated by the system using formulas that incorporate any or all of the following data: CGE Data of the user (including Training Stimulus Data and the associated Training Behavioral Response Input Data), other data associated with the user excluding CGE Data, the CGE Data of other users, the data of other users excluding CGE Data, and the data of non-users of the system or any other available data or information (referred to herein generally as “CGE Parameters Recommendations”).

The system can be configured to transmit CGE Parameters Recommendations to the Service Provider at specific time intervals or at any time as requested by the Service Provider via software that establishes a communication link with the Database combined with a computer user interface presented to the Service Provider to input configuration settings with respect to the generation of CGE Parameters Recommendations.

The system provides for application of algorithms, including machine learning algorithms that internalize the CGE Data of the user (including Training Stimulus Data and the associated Training Behavioral Response Input Data), other data associated with the user excluding CGE Data, the CGE Data of other users, the data of other users excluding CGEData, and the data of non-users of the system or any other available data or information, to programmatically refine and/or create CGE Parameters Recommendations for deployment by the system.

The Service Provider from time to time inputs and/or transmits CGE Parameters to the Controller with respect to the user based in whole or in part on the Service Provider's interaction with the user including based on the Service Provider's assessment of the user and/or the behavior of the user in response to therapy and/or training conducted by the Service Provider.

The Service Provider from time to time inputs and/or transmits CGE Parameters to the Controller with respect to the user based in whole or in part on the Service Provider's review and/or analysis of CGE Data collected with respect to the user including Training Stimulus Data and the associated Training Behavioral Response Input Data and Training Physiological Response Input Data (which may be in the form of reports generated by the Service Provider's use of the system).

The Service Provider from time to time inputs and/or transmits CGE Parameters to the Controller with respect to the user based in whole or in part on the Service Provider's review and/or analysis of recommended CGE Parameters generated by the system using formulas that incorporate any or all of the following data: CGE Data of the user (including Training Stimulus Data and the associated Training Behavioral Response Input Data and Training Physiological Response Input Data), other data associated with the user excluding CGE Data, the CGE Data of other users, the data of other users excluding CGE Data, and the data of non-users of the system or any other available data or information (i.e., the CGE Parameters Recommendations).

The system can be configured to transmit CGE Parameters Recommendations to the Service Provider at specific time intervals or at any time as requested by the Service Provider via software that establishes a communication link with the Database combined with a computer user interface presented to the Service Provider to input configuration settings with respect to the generation of CGE Parameters Recommendations.

In some embodiments, the Service Provider from time to time inputs and/or transmits CGE Parameters to the Controller with respect to the user based in whole or in part on the Service Provider's interaction with the user including based on the Service Provider's assessment of the user and/or the behavior of the user in response to therapy and/or training conducted by the Service Provider. In other embodiments, the Service Provider from time to time inputs and/or transmits CGE Parameters to the Controller with respect to the user based in whole or in part on the Service Provider's review and/or analysis of CGE Data collected with respect to the user including Training Stimulus Data and the associated Training Behavioral Response Input Data and Training Physiological Response Input (Multiple Data Streams) Data (which may be in the form of reports generated by the Service Provider's use of the system). The Service Provider may also input and/or transmit CGE Parameters to the Controller with respect to the user based in whole or in part on the Service Provider's review and/or analysis of recommended CGE Parameters generated by the system using formulas that incorporate any or all of the following data: CGE Data of the user (including Training Stimulus Data and the associated Training Behavioral Response Input Data and Training Physiological Response Input (Multiple Data Streams) Data), other data associated with the user excluding CGE Data, the CGE Data of other users, the data of other users excluding CGE Data, and the data of non-users of the system or any other available data or information (i.e., the CGE Parameters Recommendations).

The system can be configured to transmit CGE Parameters Recommendations to the Service Provider at specific time intervals or at any time as requested by the Service Provider via software that establishes a communication link with the Database combined with a computer user interface presented to the Service Provider to input configuration settings with respect to the generation of CGE Parameters Recommendations.

In some embodiments, the system provides for application of algorithms, including machine learning algorithms that internalize the CGE Data of the user (including Training Stimulus Data and the associated Training Behavioral Response Input Data and Training Physiological Response Input (Multiple Data Streams) Data), other data associated with the user excluding CGE Data, the CGE Data of other users, the data of other users excluding CGE Data, and the data of non-users of the system or any other available data or information, to programmatically refine and/or create CGE Parameters Recommendations for deployment by the system.

In one example of the invention, the CEGS comprises a computer (referred to below as the “CEGS Computer”), computer monitor, audio speakers, and a video game controller (e.g., an Xbox controller). An Eye Tracker device is mounted on the monitor and is connected to the CEGS Computer, for example, via USB or Bluetooth connection. The Controller 1 and Database 6 are maintained on the CEGS Computer. The CEGS generates a CGE comprising a computer video game that is designed to train children with Autism Spectrum Disorder to improve eye contact during social interactions by including in gameplay visual presentations of simulated social interactions with game characters as part of the CGE. In this case, the Training Stimulus is represented by different VTAs overlaying all or a portion of the face of certain game characters which are presented to the player in different visual presentations. The player is prompted to view each VTA using a visual indicator of the VTA as a Training Stimulus Response Prompt in the form of a graphical representation of the boundaries of each VTA which is presented to the player along with character dialogue during each visual presentation. For example, in some embodiments, a dotted line is used to designate the boundaries of the VTA as the visual indicator. In other embodiments, other representations may be used (e.g., shading or blurring of regions outside of the boundaries) as the visual indicator.

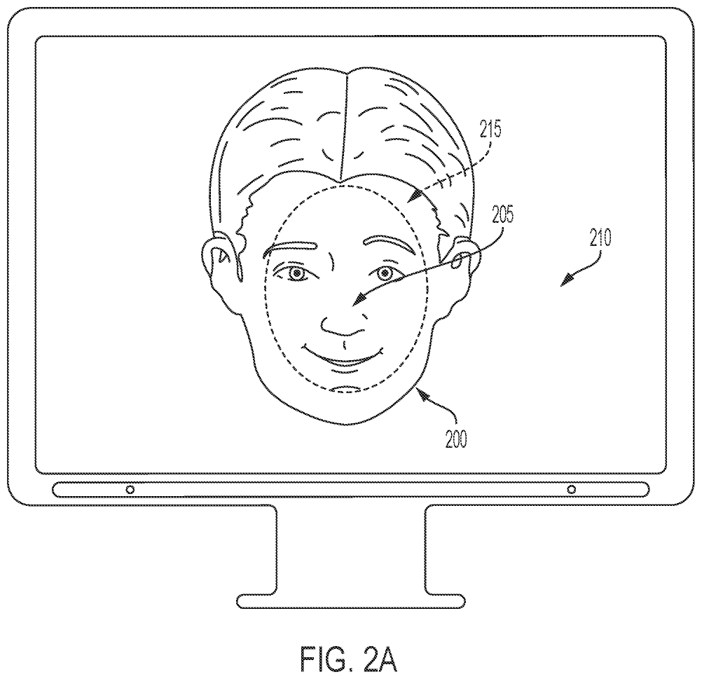

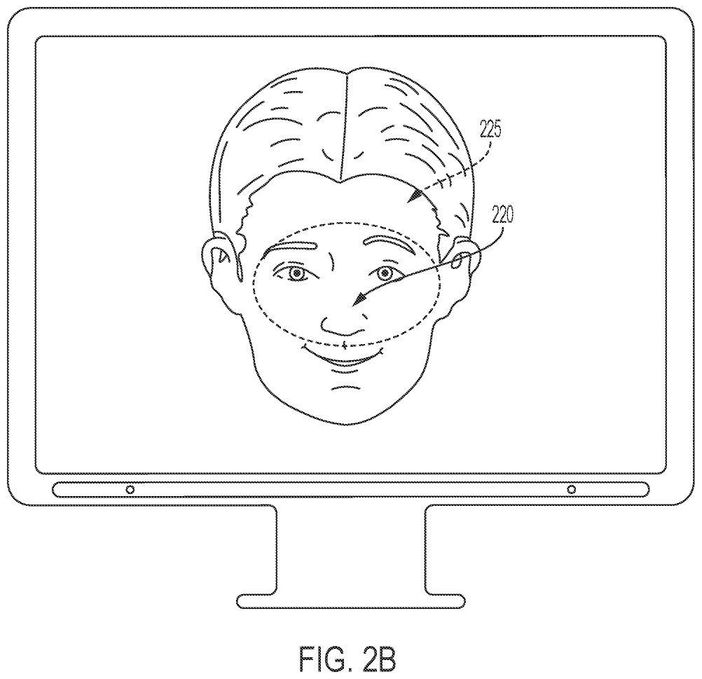

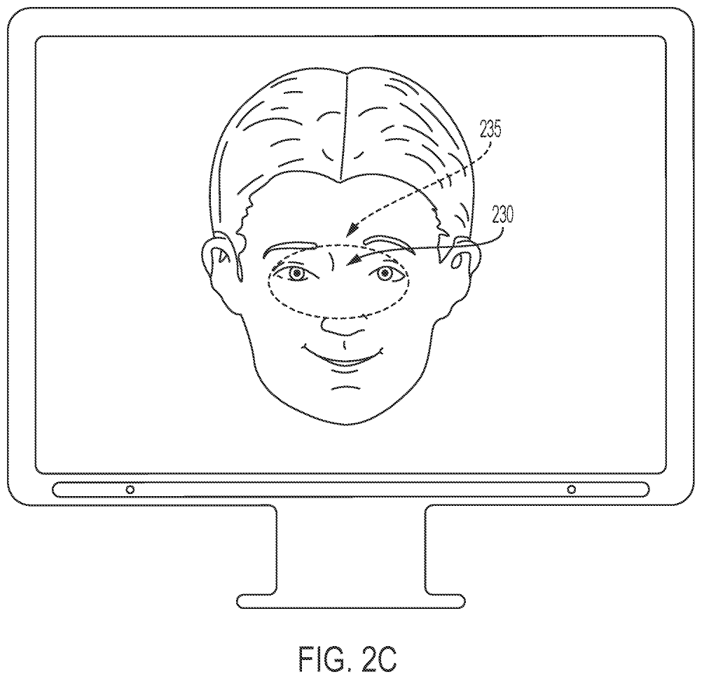

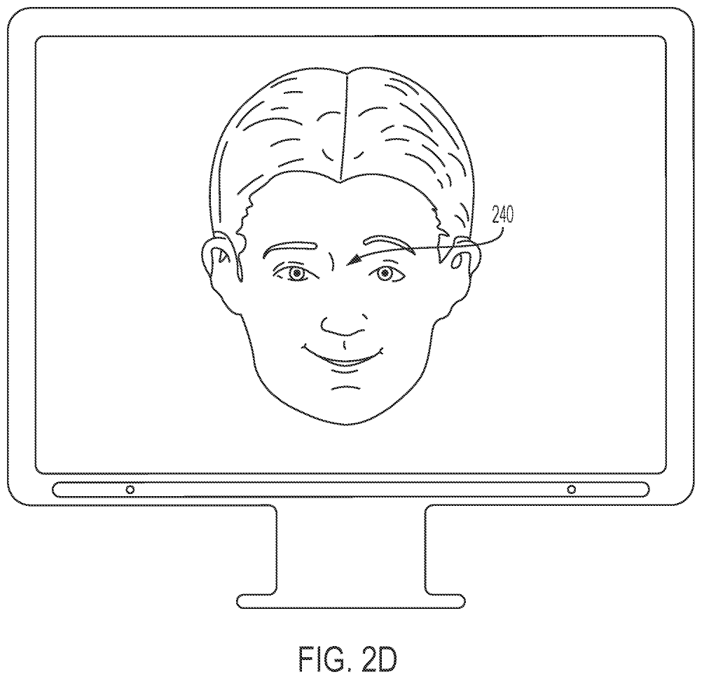

As an example, a behavioral psychologist or other attendant may serve as the Service Provider 14 and input certain CGE Parameters to the Controller including the type of the VTAs to be presented during each visual presentation, which in this case range in difficulty from the entire face of the game character with a prompt in the form of a visual indicator of the VTA to the upper half of the face of the game character with a prompt in the form of a visual indicator of the VTA to just the eyes of the game character with no prompt in the form of a visual indicator of the VTA which is illustrated in FIGS. 2A through 2D.

The Service Provider inputs CGE Parameters with respect to some or all of the VTA sequences presented to the player during gameplay including the player's required time to make initial visual contact with the VTA, required time to maintain continuous visual contact within the VTA, permissible time to stop and then resume visual contact with the VTA (deviation tolerance), shape of the VTA, size of the VTA, number of sequential repetitions involving VTA gameplay during a designated segment of time (collectively, “VTA Attributes”).

The Service Provider inputs CGE Parameters that determine the sequence of introduction of the different Training Stimulus Response Prompts and associated VTAs (including with the same or different VTA Attributes) that are introduced during gameplay. The Service Provider configures these CGE Parameters so that they are based on the Visual Gaze Performance Input Data of the player associated with the VTA sequence immediately preceding presentation of the current VTA sequence to the player.

The Service Provider also inputs CGE Parameters that alter the game experience (other than with respect to the VTAs) such as action events, game elements, and game environments for purposes including maintaining and optimizing engagement of the player. These CGE Parameters can be based on any combination of the player's CGE Data (including the ET Data, VTA Data, Visual Gaze Performance Input Data, and CGE Behavioral Performance Input Data) transmitted to the Controller during the current gameplay session by the CEGS or transmitted by the Database from a prior gameplay session. In this example, the Service Provider Individual inputs different CGE Parameters that direct the speed and number of asteroids presented per minute to the player during an asteroid shooting phase of the game and is based on CGE Behavioral Performance Input Data comprised of the player's proficiency in destroying asteroids during the previous asteroid shooting phase of the game.

While the user engages in gameplay, the system collects CGE Data, the Controller transmits the CGE Commands to the CEGS which executes on those commands in real time altering the CGE and introducing different visual presentation as the user engages in gameplay. The result is a computer game that intelligently adapts the player's game experience to achieve the optimal therapeutic effect as the player's Visual Gaze Performance Input becomes more proficient over time while using CGE Behavioral Performance Input Data to maintain player engagement.

As a second example, the system described in Example 1 may be varied to use an ECG device to transmit heart rate data to the Controller while the player engages in gameplay. The Service Provider inputs CGE Parameters that determine the sequence of introduction of the different Training Stimulus Response Prompts and associated VTAs (including with the same or different VTA Attributes) that are introduced during gameplay. The Service Provider configures these CGE Parameters so that they are based on both the (i) Visual Gaze Performance Input Data of the player, and (ii) the Singular Physiologic Data Stream Data of the player (which in this case is comprised of ECG derived heart data values or value ranges), associated with the VTA sequence immediately preceding presentation of the current VTA sequence to the player.

In this example, the Service Provider also inputs different CGE Parameters that direct the speed and number of asteroids presented per minute to the player during an asteroid shooting phase of the game and is based on both (i) CGE Behavioral Performance Input Data comprised of the player's proficiency in destroying asteroids during the previous asteroid shooting phase of the game, and (ii) the Singular Physiologic Data Stream Data of the player comprised of ECG derived heart data values or value ranges occurring during the same period of time.

While the user engages in gameplay, the system collects CGE Data and the Controller transmits the CGE Commands to the CGES which executes on those commands in real time altering the CGE as the user engages in gameplay. The result is a computer game that intelligently adapts the player's game experience to achieve the optimal training effect by applying CGE Parameters to the Visual Gaze Performance Input Data of the player as it changes over time including to increase the level of difficulty of the VTA sequence as the player's Visual Gaze Performance Input Data reflects greater player proficiency over time, (ii) applying CGE Parameters to CGE Behavioral Performance Input Data to maintain player engagement, and (iii) uses CGE Parameters applied to the Singular Physiologic Data Stream Data to achieve biofeedback like functionality to train the player to reach and/or maintain a targeted physiological state (which in this case is in the form of a certain heart rate derived value range) during specified VTA sequences and/or at other times including during general gameplay.

In a third example, the system described in one or more of the examples discussed above may be varied to use an EEG device to measure electrical brain activity and further use a GSR device to measure galvanic skin resistance activity while the player engages in gameplay. The Service Provider inputs CGE Parameters that determine the sequence of introduction of the different Training Stimulus Response Prompts and associated VTAs (including with the same or different VTA Attributes) that are introduced during gameplay. The Service Provider configures these CGE Parameters so that they are based on the: (i) Visual Gaze Performance Input Data of the player, and (ii) the Multiple Physiologic Data Streams Data of the player (which in this case is comprised of ECG derived heart data values or value ranges, EEG and GSR data values or value ranges), associated with the VTA sequence immediately preceding presentation of the current VTA sequence to the player, and (iii) the CGE Behavioral Performance Input Data comprised of the player's proficiency in making game controller based selections that match the emotion of the game character presented during the current VTA sequence which in this example represents a second training function of the system.

In this example, the Service Provider also inputs different CGE Parameters that direct the speed and number of asteroids presented per minute to the player during an asteroid shooting phase of the game and is based on both (i) CGE Behavioral Performance Input Data comprised of the player's proficiency in destroying asteroids during the previous asteroid shooting phase of the game, and (ii) the Multiple Physiologic Data Streams Data of the player (which in this case is comprised of ECG derived heart data values or value ranges, EEG and GSR data values or value ranges) occurring during the same period of time.

While the user engages in gameplay, the system collects CGE Data and the Controller transmits the CGE Commands to the CGES which executes on those commands in real time altering the CGE as the user engages in gameplay. The result is a computer game that intelligently adapts the player's game experience to achieve the optimal training effect by applying CGE Parameters to the Visual Gaze Performance Input Data of the player as it changes over time including the ability to increase the level of difficulty of the VTA sequence as the player's Visual Gaze Performance Input Data reflects greater player proficiency over time, (ii) applying CGE Parameters to the Multiple Physiologic Data Streams Data of the player to achieve biofeedback like functionality to train the player to reach and/or maintain a targeted physiological state during specified VTA sequences, (iii) applying CGE Parameters to the CGE Behavioral Performance Input Data to perform a second training function in the form of game character emotion recognition, and (iv) applying CGE Parameters to the CGE Behavioral Performance Input Data and Multiple PhysiologicData Streams Data to maintain player engagement (in this example, during the asteroid shoot phase of the game) over time.

In another example, the system described in one or more of the examples discussed above may be modified to use a communication link or links established over a public computer network, private computer network, or over the Internet between the Database and sources of data (“Data Sources”) that include both CGE Data and non-CGE Data of other users of the system, the data of non-users of the system, and any other available data or information (“Other User and Non-User Data”) where such Data Sources can include:

a computer used by a second user of the system while such second user is engaged in a CGE, (ii) a second database used to store and transmit the Other User and Non-User Data including any or all such data collected prior to the user's then current use of the system and/or collected concurrently with the user's then current use of the system, and/or (iii) data acquired through automated intelligently targeted internet and/or database searches of relevant research.

In some embodiments, the Controller Operator is the combination of a Service Provider that manually inputs CGE Parameters, and software that programmatically enters CGE Parameters through application of algorithms, including machine learning algorithms that internalize the CGE Data of the user (including the user's current and/or prior Multiple Physiologic Data Streams Data including the Training Physiological Response Input (Multiple Data Streams) Data), other data associated with the user excluding CGE Data, and the Other User and Non-User Data, to programmatically refine and/or create new CGE Parameters.

The algorithms, including machine learning algorithm continually attempts to optimize the CGE Parameters to maximize improvements in user Visual Gaze Performance Input. To do so, the algorithm continually estimates which parameters are most likely to maximize improvements in user Visual Gaze Performance Input based on all the available data and information, adjusting these expected optimal parameters in some way (either randomly or via some adjustment algorithm), and returning them to the CEGS. The user would then complete the CGE with the returned CGE Parameters, generating new data on which the algorithms, including machine learning algorithms could operate. Such a machine learning algorithm would likely be categorized as a “reinforcement learning” algorithm, but it could also take some other form.

In another example, reference is made to FIG. 1 to illustrate an embodiment of the invention designed to train children with autism spectrum disorder to make or increase eye contact with others during social interactions, a critical social skill.

The Service Provider 14 provides therapy to User 4, which is a child with autism.

Prior to accessing User Interface 13, Service Provider 14 assesses User's proficiency of making eye contact during social interactions.

The Service Provider 14 uses User Interface 13 which is accessed using a web browser. The Service Provider 14 creates an account for the User 4 using the User Interface 13. The Service Provider 14 enters User 4 information including, name, password, age, gender. This data is transmitted to Database 6 and is stored there for access by the system components.

The Service Provider 14, based on the Service Provider 14 assessment of User's 4 proficiency of making eye contact during social interactions (as described above), uses User Interface 13 to enter CGE Parameters, which is performed by Service Provider 14 selecting from among three different predefined groups designated as “Low”, “Medium”, and “High”, each group comprising a unique set of CGE Parameters (the “Skill Ratings Parameters”). This data is transmitted to Controller Operator—Individual 11, which is a software designed for individuals to enter and/or modify CGE Parameters.

When the training session is initiated, the Controller 1 sends CGE Commands to CEGS 2, which presents the User 4 with Other Prompt 35 for User 4 to enter their user name and password. When the User 4 enters the prompted information using Keyboard 532, this CGE Behavioral Performance Input 503 is transmitted to the Controller 1 which validates the user credentials using the data in the Database 6.

Upon successful validation of user credentials using the validation process described above, the Controller 1 accesses User's 4 data stored in Database 6 and retrieves CGE Parameters from Controller Operator—Individual 11 and uses this information to compute and sends CGE Commands to CEGS 2. Upon receiving CGE Commands from Controller 1, CEGS 2 initiates a CGE 3, which in this example is comprised of a computer, monitor, software, audio speakers and video game controller (e.g. Xbox controller), that initiates a video game which is comprised of a series of CGEs 3 and associated visual presentations, including CGEs 3 that require User 4 to gaze within specific VTAs 34.

A commercial Eye Tracker 511 is mounted below the monitor and is connected to Controller 1 via USB. The Controller 1 also has necessary software to capture all data generated by the devices connected to it, and in this example, Controller 1 has the necessary software to capture ET Data 501 and Visual Gaze Performance (“VGP”) Input 500 data generated by Eye Tracker 511.

The game includes User's 4 interactions with game characters during visual presentations. During these game character interactions, a Training Stimulus 31 is presented to the User 4 in the form of a visual display of the game character's face presenting game dialog in audio form During a first game sequence a Training Stimulus Response Prompt 32 is displayed to the User 4 in the form of a graphical display of a perimeter of the VTA 34, which in this case is an area that includes the eyes and nose of the face of the game character as illustrated in FIG. 2B. This represents a single training repetition.

User 4 responds to the Training Stimulus Response Prompt 32 which may include either looking at or not looking at the area within the VTA 34.

The Eye Tracker 511 coupled with necessary software captures the User's 4 VGP Input 500 as a response to presentation of VTA 34 (the Training Stimulus Response Prompt 32) and transmits this CGE Data to Controller 1.

Upon receiving CGE Data, Controller 1 first determines if there is an association between the VTA 34 (the Training Stimulus Response Prompt 32) and User's 4 VGP Input 500 data. Controller 1 may use an internal and/or external PMD Synchronization software and/or internal logic to associate this data. Controller 1 then performs a “first validation step” wherein Controller 1 validates this data against applicable preconfigured CGE Parameters and applicable CGE Parameters configured by the Service Provider 14 which in this example may include the Skill Ratings Parameters. In this example, the applicable preconfigured CGE Parameters include the user's required time to make initial visual contact with the VTA, required time to maintain continuous visual contact within the VTA, permissible time to stop and then resume visual contact with the VTA (deviation tolerance).

If the CGE Data passes the first validation step, Controller 1 sends a CGE Command to CEGS 2 to generate a second training repetition using the process previously described for generation of the first training repetition with the possible additional step of use of different CGE Parameters, (including based on CGE Data collected during the first repetition and/or following the first repetition including in the event of a first validation step failure, as described in the next step), in the generation of the second training repetition.

If the CGE Data fails the first validation step, Controller 1 sends a CGE Command to CEGS 2 to generate a second game character which provided instructions and encouragement to User 4 to engage in the targeted behavior, which in this case is making a visual contact within the VTA in conformance with the associated CGE Command Parameters. Following this CGE, the Controller 1 sends CGE Commands to CEGS 2 to generate a second training repetition as described above.

Controller 1 determines the maximum number of training repetitions within a single training sequence based upon preconfigured CGE Parameters and/or Service Provider 14 defined CGE Parameters.

During a second game sequence, Controller 1 presents a Training Stimulus Response Prompt 32 to the User 4 in the form of a graphical display of a perimeter of the VTA 34 different from that which was presented during the last repetition of the first game sequence, which in this case is the eye region only of the face of the game character as illustrated in FIG. 2C representing a potentially more challenging task for User 4.

All data transmitted to Controller 1 during these game sequences is saved to Database 6. At any time, Service Provider 14 (using User Interface 13) can generate reports against any data stored in the Database 6.

In this next example, reference is made to FIG. 1 to illustrate an embodiment of the invention designed to train children with autism spectrum disorder to make or increase eye contact with others during social interactions, and recognize or increase recognition of the emotions of others during social interactions, two critical social skills.

Service Provider 14 provides therapy to User 4, which is a child with autism Prior to accessing User Interface 13, Service Provider 14 assesses User's 4 proficiency of making eye contact and recognizing the emotions of others during social interactions.

The Service Provider 14 uses User Interface 13 which is accessed using a web browser. The Service Provider 14 creates an account for the User 4 using the User Interface 25. The Service Provider 14 enters User 4 information including, name, password, age, gender. This data is transmitted to Database 6 and is stored there for access by the system components.

The Service Provider 14, based on the assessment of User's 4 proficiency of making eye contact (“skill 1”) and recognizing the emotions of others (“skill 2”) during social interactions as described above, uses User Interface 13 to enter CGE Parameters for skill 1 and skill 2, which is performed by Service Provider 14 selecting from among three different predefined groups for each of skill 1 and skill 2 designated as “Low”, “Medium”, and “High”, each group comprising a unique set of CGE Parameters, with a separate selection made for each of skill 1 and skill 2 (collectively the “Skills Ratings Parameters”). This data is transmitted to Controller Operator—Individual 11, which is a software designed for individuals to enter and/or modify CGE Parameters.

When the training session is initiated, the Controller 1 sends CGE Commands to CEGS 2, which presents the User 4 with Other Prompts 35 for User 4 to enter their user name and password. When the User 4 enters the prompted information using Keyboard 532, this CGE Behavioral Performance Input 503 is transmitted to the Controller 1 which validates the user credentials using the data in the Database 6.

Upon successful validation of user credentials using the validation process described above, Controller 1 accesses User's 4 data stored in Database 6 and retrieves CGE Parameters from Controller Operator—Individual 11 and uses this information to compute and sends CGE Commands to CEGS 2. Upon receiving CGE Commands from Controller 1, CEGS 2 initiates a CGE 3, which in this example is comprised of a computer, monitor, software, audio speakers and video game controller (e.g., Xbox controller), that initiates a video game which is comprised of a series of CGEs 3 and associated visual presentations, including CGEs 3 that require User 4 to gaze within specific VTAs 34.

A commercial Eye Tracker 511 is mounted below the monitor and is connected to Controller 1 via USB. The Controller 1 also has necessary software to capture all data generated by the devices connected to it, and in this example, Controller 1 has the necessary software to capture ET Data 501 and VGP Input 500 data generated by Eye Tracker 511.

The game includes User's 4 interactions with game characters during visual presentations. During these game character interactions, a Training Stimulus 31 is presented to the User 4 in the form of a visual presentation of a game character's face (which is blurred) presenting game dialog in audio form and images of people expressing different emotions with the corresponding labels of such emotion presented in text form below each image and a unique letter in text form of one of the Game Controller 533 buttons (“Emotion Matching Images and Text”). During a first game sequence a Training Stimulus Response Prompt 32 is displayed to User 4 in the form of a VTA 34, which in this case is the blurred face of the game character.

User 4 responds to the Training Stimulus Response Prompt 32 which may include either looking at or not looking at the area within the VTA 34.

The Eye Tracker 511 coupled with necessary software captures the User's 4 VGP Input 500 as a response to presentation of VTA 34 (the Training Stimulus Response Prompt 32) and transmits this CGE Data to Controller 1.

Upon receiving CGE Data, Controller 1 first determines if there is an association between the VTA 34 (the Training Stimulus Response Prompt 32) and the User's 4 VGP Input 500 data. Controller 1 may use an internal and/or external PMD Synchronization software and/or internal logic to associate this data. Controller 1 then performs a “first validation step” wherein Controller 1 validates this data against applicable preconfigured CGE Parameters and applicable CGE Parameters configured by the Service Provider 14 which in this example may include the Skills Ratings Parameters. In this example, the applicable preconfigured CGE Parameters include the user's required time to make initial visual contact with the VTA, required time to maintain continuous visual contact within the VTA, permissible time to stop and then resume visual contact with the VTA (deviation tolerance) and time permitted for user response to all Training Stimuli Response Prompts 3.

If the CGE Data fails the first validation step, Controller 1 sends a CGE Command to CEGS 2 to generate a second game character which provided instructions and encouragement to User 4 to engage in the targeted behavior, which in this case is making a visual contact within the VTA 34 in conformance with the associated CGE Command Parameters. Following this CGE, the Controller 1 sends CGE Commands to CEGS 2 to repeat the training sequence.

If the CGE Data passes the first validations step, Controller 1 sends a CGE Command to CEGS 2 to remove the blurring of game character's face.

Controller 1 then sends CGE Commands to CEGS 2 to transmit a Training Stimulus Response Prompt 32 to prompt User 4 to match the game character's emotion with the matching emotion displayed among the set of images in the Emotion Matching Images and Text by pressing the Game Controller 533 button with the same letter as presented for the corresponding image within the Emotion Matching Images and Text. Upon User 4 Game Controller 533 button selection, this CGE Behavioral Performance Input Data 503 is transmitted to Controller 1.

Upon receiving CGE Data, Controller 1 first determines if there is an association between the Training Stimulus Response Prompt 32 and the User 4 CGE Behavioral Performance Input Data 503. Controller 1 may use an internal and/or external PMD Synchronization software and/or internal logic to associate this data. Controller 1 then performs a “second validation step” wherein Controller 1 then validates this data against applicable CGE Parameters configured by the Service Provider 14 which in this example may include the Skills Ratings Parameters and applicable preconfigured CGE Parameters. In this example, the applicable preconfigured CGE Parameters is the correct letter of the Game Controller 533 button.

If the CGE Data fails the second validation step, Controller 1 sends a CGE Command to CEGS 2 to generate a second game character which provided instructions and encouragement to User 4 to engage in the targeted behavior, which in this case is making the appropriate selection from the Emotion Matching Images and Text by pressing the correct letter of the Game Controller 533 button. Following this CGE, the Controller 1 sends CGE Commands to CEGS 2 to repeat the training sequence.

If the CGE Data passes the second validation step Controller 1 sends CGE Commands to CEGS 2 to generate a second training repetition using the process previously described for generation of the first training repetition, which in addition, may include as an additional step, use of different CGE Parameters (including based on CGE Data collected during the first repetition sequence or first repetition sequences in the event of occurrence of validation failures during the first repetition sequence), in the generation of the second training repetition.

Controller 1 determines the maximum number of training repetitions within a single training sequence based upon preconfigured CGE Parameters and/or Service Provider 14 defined CGE Parameters.

During a second game sequence the process is modified so that instead of the removal of blurring of the entire face of game character, removal of blurring is limited to the upper half of the game character's face, representing a potentially more challenging task for User 4.

At any time, Service Provider 14 (using User Interface 13) can generate reports against any data stored in the Database 6.

In this next example, reference is made to FIG. 1 to illustrate an embodiment of the invention designed to train children with autism spectrum disorder to make or increase eye contact with others during social interactions, and recognize or increase recognition of the emotions of others during social interactions, two critical social skills, and improve their emotional state during social interactions.

Service Provider 14 provides therapy to User 4, which is a child with autism Prior to accessing User Interface 13, Service Provider 14 assesses User's 4 proficiency of making eye contact, recognizing the emotions of others, and level of anxiety during social interactions.

The Service Provider 14 uses User Interface 13 which is accessed using a web browser. The Service Provider 14 creates an account for the User 4 using the User Interface 13. The Service Provider 14 enters User 4 information including, name, password, age, gender. This data is transmitted to Database 6 and is stored there for access by the system components.

The Service Provider 14, based on the assessment of User's 4 proficiency of making eye contact (“skill 1”), recognizing the emotions of others (“skill 2”) and level of anxiety during social interactions (“behavior 1”), uses User Interface 13 to enter CGE Parameters for skill 1 and skill 2 which is performed by Service Provider 14 selecting from among three different predefined groups for each of skill 1 and skill 2 designated as “Low”, “Medium”, and “High”, each group comprising a unique set of CGE Parameters with a separate selection made for each of skill 1 and skill 2 (collectively the “Skills Ratings Parameters”), and Service Provider 14 further enters into User Interface 13 separate High to Low values to define acceptable value ranges for each of three physiological measures, EEG 521, ECG 522, and GSR 523 (collective referred to as “Acceptable Physiological Value Ranges”). This data is transmitted to Controller Operator—Individual 11, which is a software designed for individuals to enter and/or modify CGE Parameters.

Prior to beginning the training session, the following three PMDs 52 are applied to the body of User 4: ECG measuring device 522, GSR measuring device 523, and EEG measuring device 521, which are connected to Controller 1 via Bluetooth data link or USB wired connection.

Prior to beginning the training session, the following three PMDs 52 are applied to the body of User 4: ECG measuring device 522, GSR measuring device 523, and EEG measuring device 521, which are connected to Controller 1 via Bluetooth data link or USB wired connection.

When the training session is initiated, the Controller 1 sends CGE Commands to CEGS 2, which presents the User 4 with Other Prompt 35 for User 4 to enter their user name and password. When the User 4 enters the prompted information using Keyboard 532, this CGE Behavioral Performance Input 503 is transmitted to the Controller 1 which validates the user credentials using the data in the Database 6.

Upon successful validation of user credentials using the validation process described above, the Controller 1 accesses User's 4 data stored in Database 6 and retrieves CGE Parameters from Controller Operator—Individual 11 and uses this information to compute and sends CGE Commands to CEGS 2. Upon receiving CGE Commands from Controller 1, CEGS 2 initiates a CGE 3, which in this example is comprised of a computer, monitor, software, audio speakers and video game controller (e.g. Xbox controller), that initiates a video game which is comprised of a series of CGEs 3 and associated visual presentations, including CGEs 3 that require User 4 to gaze within specific VTAs 34.

A commercial Eye Tracker 511 is mounted below the monitor and is connected to Controller 1 via USB. The Controller 1 also has necessary software to capture all data generated by the devices connected to it, and in this example, Controller 1 has the necessary software to capture ET Data 501 and VGP Input 500 data generated by Eye Tracker 511, and Multiple Physiological Data Streams (“MPDS”) 502 data generated by PMDs 52. MPDS 502 data is collected and continuously transmitted to Controller 1 in near real time during the entire training session.

The game includes User's 4 interactions with game characters. During these game character interactions, a Training Stimulus 31 is presented to User 4 in the form of a visual display of a game character's face (which is blurred) presenting game dialog in audio form and images of people expressing different emotions with the corresponding labels of such emotion presented in text form below each image and a unique letter in text form of one of the Game Controller 533 buttons (“Emotion Matching Images and Text”). During a first game sequence a Training Stimulus Response Prompt 32 is displayed to the User 4 in the form of a VTA 34, which in this case is the blurred face of the game character.

User 4 responds to the Training Stimulus Response Prompt 32 which may include either looking at or not looking at the area within the VTA 34.

The Eye Tracker 511 coupled with necessary software captures the User's 4 VGP Input 500 as a response to presentation of VTA 34 (the Training Stimulus Response Prompt 30-32) and transmits this CGE Data to Controller 1.

Upon receiving CGE Data, Controller 1 first determines if there is an association between the VTA 34 (the Training Stimulus Response Prompt 32) and the User's 4 VGP Input 500 data. Controller 1 also looks at the MPDs 502 collected for the time period starting from introduction of Training Stimulus Response Prompt 32 and ending upon User's 4 responses. Controller 1 may use an internal and/or external PMD Synchronization software and/or internal logic to associate this data. Controller 1 then performs a “first validation step” wherein Controller 1 validates this data against applicable preconfigured CGE Parameters and applicable CGE Parameters configured by the Service Provider 14 which in this example may include the Skills Ratings Parameters and includes the Acceptable Physiological Value Ranges. In this example, the applicable preconfigured CGE Parameters include the user's required time to make initial visual contact with the VTA, required time to maintain continuous visual contact within the VTA, permissible time to stop and then resume visual contact with the VTA (deviation tolerance), time permitted for user response to all Training Stimuli Response Prompts 3 (“Required User Response Time”).

If the CGE Data fails the first validation step, Controller 1 sends a CGE Command to CEGS 2 to generate a second game character which provided instructions and encouragement to User 4 to engage in the targeted behavior. For example, if the validation fails due to failure to make visual contact within the VTA, the second game character will encourage the targeted behavior of making a visual contact within the VTA. If validation fails due to PMD 52 measurements that fall outside of the Acceptable Physiological Value Ranges, the second game character will encourage behavior targeted to affect changes in physiology, such as deep breathing and visualization techniques to induce a more relaxed state and mental focus. Following this CGE, the Controller 1 sends CGE Commands to CEGS 2 to repeat the training sequence.

If the CGE Data passes the first validations step, Controller 1 sends a CGE Command to CEGS 2 to remove the blurring of game character's face.

Controller 1 then sends CGE Commands to CEGS 2 to transmit a Training Stimulus Response Prompt 32 to prompt User 4 to match the game character's emotion with the matching emotion displayed among the set of images in the Emotion Matching Images and Text by pressing the Game Controller 533 button with the same letter as presented for the corresponding image within the Emotion Matching Images and Text. Upon User 4 Game Controller 533 button selection, this CGE Behavioral Performance Input Data 503 is transmitted to Controller 1.

Upon receiving CGE Data, Controller 1 first determines if there is an association between the Training Stimulus Response Prompt 32 and the User's 4 CGE Behavioral Performance Input Data 503. Controller 1 also looks at the MPDs 502 collected for the time period starting from introduction of Training Stimulus Response Prompt 32 and ending upon User's 4 responses. Controller 1 may use an internal and/or external PMD Synchronization software and/or internal logic to associate this data. Controller 1 then performs a “second validation step” wherein Controller 1 validates this data against applicable CGE Parameters configured by the Service Provider 14 and applicable preconfigured CGE Parameters. In this example, the applicable preconfigured CGE Parameters is the correct letter of the Game Controller 533 button and the applicable CGE Parameters configured by the Service Provider 14 are the Acceptable Physiological Value Ranges.

If the CGE Data fails the second validation step because the incorrect letter was selected on the Game Controller 533, Controller 1 sends a CGE Command to CEGS 2 to generate a second game character to provide instruction and encouragement to User 4 to engage in the targeted behavior, which in this case is making the appropriate selection from the Emotion Matching Images and Text by pressing the correct letter of the Game Controller 533 button. If the CGE Data fails the second validation step due to PMD 52 measurements that fall outside of the Acceptable Physiological Value Ranges, the second game character will encourage behavior targeted to affect changes in physiology, such as deep breathing and visualization techniques to induce a more relaxed state and mental focus. Following this CGE, the Controller 1 sends CGE Commands to CEGS 2 to repeat the training sequence.

If the CGE Data passes the second validation step, Controller 1 sends CGE Commands to CEGS 2 to generate a second training repetition using the process previously described for generation of the first training repetition, which in addition, may include as an additional step, use of different CGE Parameters (including based on CGE Data collected during the first repetition sequence or first repetition sequences in the event of occurrence of validation failures during the first repetition sequence), in the generation of the second training repetition.

Controller 1 determines the maximum number of training repetitions within a single training sequence based upon preconfigured CGE Parameters and/or Service Provider 14 defined CGE Parameters.

During a second game sequence the process is modified so that instead of the removal of blurring of the entire face of game character, removal of blurring is limited to the upper half of the game character's face, representing a potentially more challenging task for User 4.

At any time, Service Provider 14 (using User Interface 13) can generate reports against any data stored in the Database 6.

In this next example, reference is made to FIG. 1 to illustrate an embodiment of the invention designed to train children with autism spectrum disorder in any one or more of the previously discussed skills, making or increasing eye contact with others during social interactions, and recognizing or increasing recognition of the emotions of others during social interactions, two critical social skills, and improvement of their emotional state during social interactions.

In all of the embodiments described herein, the use of a commercial Eye Tracker 511 mounted below the monitor that is connected to Controller 1 via USB can be substituted for a virtual reality headset with eye tracking capability 512 that is connected to CEGS 2, so that User 4 experiences a CGE 3 in the form of a video game in a virtual reality platform. The virtual reality headset with eye tracking capability 512 is also connected to the Controller 1 and collects and transmits VGP Input Data 500 to Controller 1 using its eye tracking capabilities during transmission of the CGE 2 to User 4.

In this next example, reference is made to FIG. 1 to illustrate an embodiment of the invention designed to train children with autism spectrum disorder in any one or more of the previously discussed skills of making or increasing eye contact with others during social interactions and recognizing or increasing recognition of the emotions of others during social interactions, and foster improvement of their emotional state during social interactions through a process that uses eye tracking data to provide feedback to the user to optimize eye positioning for capture of eye tracking data.

All of the embodiments described herein can additionally include the following embodiment which provides for use of behavioral training while viewing VTA 34 to maintain the positioning of the eyes of User 4 so as to optimize the capture of complete ET Data 501 for use by the system

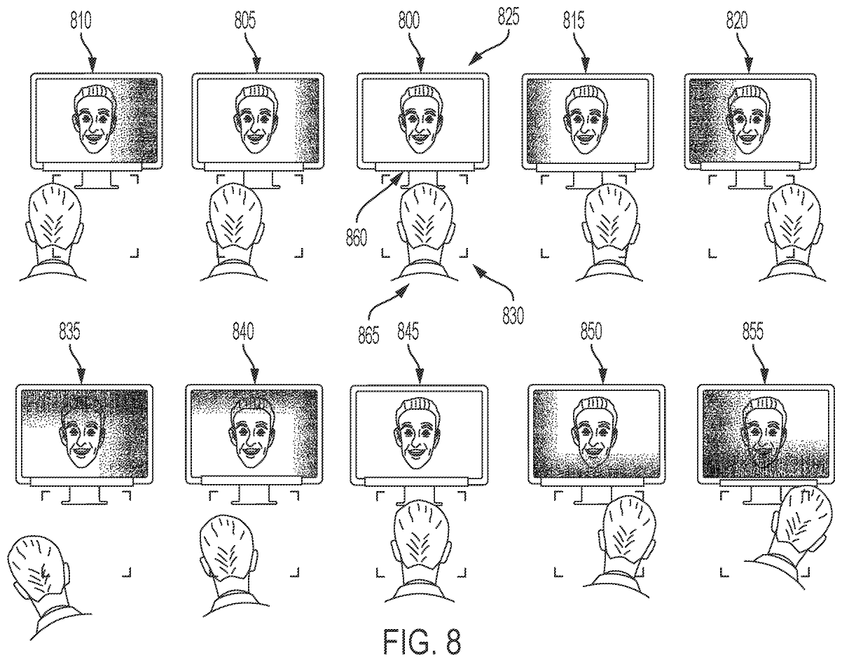

In order for Eye Tracker 511 to capture complete ET Data 501, the position of User 4 eyes in physical space in relation to the position of Eye Tracker 511 in physical space should be within a range of locations such that Eye Tracker 511 is able to capture complete ET Data 501 (“Eye Tracker Data Capture Field”). This is represented by the bracket area 830 in FIG. 8.

Controller 1 has the necessary software to capture all data generated by Eye Tracker 511 including data that indicates the position of User 4 eyes in physical space in relation to the Eye Tracker Data Capture Field where such data indicates (a) both eyes are positioned completely outside of the Eye Tracker Data Capture Field, (b) one eye is positioned completely outside of the Eye Tracker Data Capture Field with an indication of which eye is missing, (c) either eye or both eyes are positioned too far to the left of Eye Tracker 511, (d) either eye or both eyes are positioned too far to the right of Eye Tracker 511, (e) either eye or both eyes are positioned too close to Eye Tracker 511, (f) either eye or both eyes are positioned too far away from Eye Tracker 511, (g) either eye or both eyes are positioned too high above Eye Tracker 511, (h) either eye or both eyes are positioned too far below Eye Tracker 511, (i) both eyes are positioned within the Eye Tracker Data Capture Field (collectively, “Eyes Positioning Data”). Eyes Positioning Data is constantly generated by Controller 1 including all occurrences of (a) through (h), each such occurrence referred to as an “Eye Repositioning Required Event”.

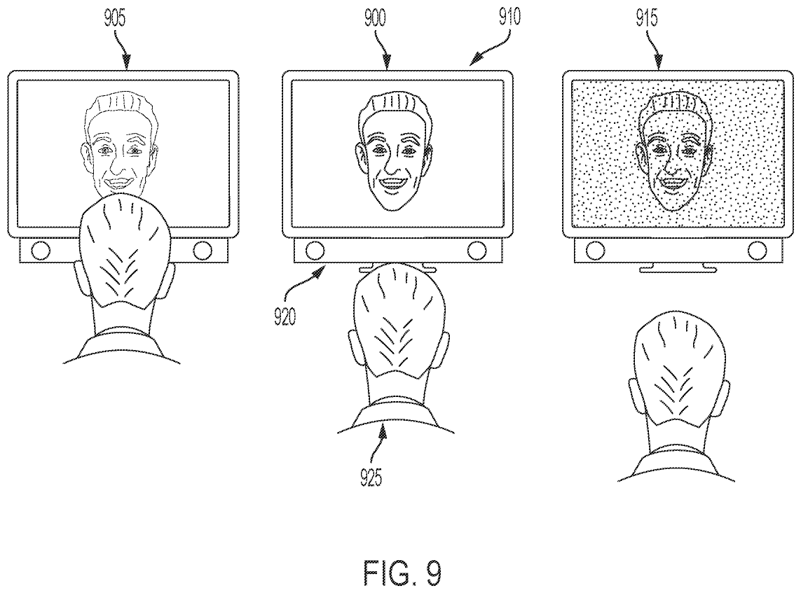

If at any time an Eyes Positioning Data is generated indicating Eye Repositioning Required Event for a constant increment of time as defined by Controller 1, Controller 1 transmits a CGE Command to CEGS 2 to generate a CGE 3 that indicates to User 4 to take an action to reposition User 4's eyes so that they are positioned within the Eye Tracker Data Capture Field (a “Reposition Instruction”). A Reposition Instruction can be in any type of form or in concurrent multiple forms capable of being generated by the CGES 2 including audio and/or visual form (which may or may not include a coding or symbol system). For example, a Reposition Instruction can take the form of changes in color, brightness, contrast, and/or clarity of a portion of or all of, a computer monitor screen, as well as, in visual form, be associated in location on the screen to the desired change in eye position, and be presented for a singular duration of time or presented until the User 4's eyes are positioned within the Eye Tracker Data Capture Field. This is illustrated in FIG. 8 and FIG. 9.

Reposition Instructions can be transmitted concurrently and present to User 4 in a manner that adaptively changes so as to create the perception to User 4 to seamlessly correspond to the degree to which User 4 changes eye position as User 4 moves closer to or farther away from the Eye Tracker Data Capture Field. For example, the Reposition Instructions can reduce the clarity of the images presented on the computer monitor as User 4 moves farther away from the Eye Tracker Data Capture Field and conversely increase the clarity of the images presented on the computer monitor as User 4 moves closer to the Eye Tracker Data Capture Field. This is illustrated in FIG. 9.

Once User 4's eyes are positioned within the Eye Tracker Data Capture Field, as a result of User 4's change in eye position for a constant increment of time as defined by Controller 1, Controller 1 determines User 4's eyes are positioned within the Eye Tracker Data Capture Field, then Controller 1 may transmit a CGE Command to CEGS 2 to generate a CGE 3 indicating to User 4 that User 4's eye position is now properly positioned (a “Reposition Confirmation”). A Reposition Confirmation can be in any type of form capable of being generated by the CGES 2 including audio and/or visual form (which may or may not include a coding or symbol system) and in multiple forms including, for example, changes in color, brightness, contrast, and/or clarity of a portion of or all of, a computer monitor screen for a singular duration of time or presented until the User 4's eyes are positioned outside the Eye Tracker Data Capture Field.

By way of further example, in the event an Eye Repositioning Required Event occurs where User 4's eyes are positioned too far to the left for a constant increment of time as defined by Controller 1, Controller 1 transmits a CGE Command to CEGS 2 to generate a CGE 3, in which Reposition Instructions take multiple concurrent forms, an audio instruction is given to User 4 to move eye position to the right while concurrently a portion of the right side of the computer monitor is visually altered so that it becomes a solid color. Reposition Instructions are incrementally generated so that as User 4 moves farther to the left more of the right side of the computer monitor becomes a solid color. Conversely, Reposition Instructions are incrementally generated so that as User 4 moves eye position to the right less of the right side of the computer monitor becomes a solid color until Controller 1, as a result of User 4 change in eye position, determines User 4 eyes are positioned within the Eye Tracker Data Capture Field. Controller 1 then transmits a CGE Command to CEGS 2 to generate a Reposition Confirmation in the form of an audio message to User 4 indicating to User 4 that User 4's eye position is now properly positioned while concurrently Controller 1 transmits a CGE Command to CEGS 2 to generate a Reposition Confirmation in visual form by removing the solid color from the right portion of the computer monitor and returning it to normal rendering of images on the full monitor screen. This is illustrated in FIG. 8.

In this next example, reference is made to FIG. 1 to illustrate an embodiment of the invention designed to apply machine learning to any type of training that has a visual training component, including those previously discussed, training children with autism spectrum disorder to make or increase eye contact with others during social interactions, recognizing or increasing recognition of the emotions of others during social interactions, and fostering improvement of their emotional state during social interactions where visual contact is normative, through use of adaptive VTAs.

In such applications, the Controller Operator-Machine 12, which may be a computer or series of computers with computing software designed to perform processes the described in this example, will apply algorithms, including machine learning algorithms (such as reinforcement learning algorithms) to a broad array of data including: (a) CGE Data of the User 4, and (b) other data associated with the User 4 excluding CGE Data (including CGE Data of other users, the data of other users excluding CGE Data, and (c) the data of non-users of the system or any other available data or information), whether accessed from Database 6 or Internet cloud services 7. This includes any or all such data collected prior to the user's then current use of the system and/or collected concurrently with the user's then current use of the system. The algorithms, including machine learning algorithms will use that data to programmatically refine and/or create CGE Parameters in order to maximize or optimize some outcome variable. In the example discussed previously, where the application is being used to train children with autism spectrum disorder to increase eye contact, the outcome variable would be the amount of eye contact being made, and the algorithms, including machine learning algorithms, would be optimizing the CGE Parameters in order to maximize the child's eye contact (or have it reach some target, optimal level).

All of the embodiments described herein can additionally include the following embodiments in which Controller 1 may use predefined CGE Parameters, CGE Parameters configured by the Service Provider 14, and/or CGE Parameters configured by Controller Operator Machine 12 as applied to Data including Visual Gaze Performance Input Data 500, Multiple Physiological Data Streams 502 and CGE Behavioral Performance Input Date 503 to present VTAs 34 in different ways as more fully described below.

The present invention contemplates VTAs are generated in a visual presentation (which can be electronically generated or in a real world environment) based on the user's gaze with respect to a first VTA as indicated by eye tracking measurement data, and may include the user's behavioral and/or physiological measurement data during presentation of the VTA as additional criteria for how the next VTA will be generated by the invention. This invention presents an infinite number of parameter combinations which the system can be configured to use based on possible combinations of that measurement data to determine how VTAs will be presented. The invention also provides for an infinite number of ways in which VTAs can be presented by virtue of the fact that VTAs can be presented in different forms that vary widely, including vary by size, shape, location, speed of presentation, duration of presentation, inclusion of prompt, etc. and overlay all or any portion of any type of visual presentation. The following are used to illustrate a small number of these possible embodiments.

FIGS. 2A-2D illustrate an example of narrowing a VTA in response to collected measurement data, according to some embodiments. Starting with FIG. 2A, a human face 200 is presented in a visual presentation such as a movie or video game which may be presented as a simulation of a social interaction with a single individual. A first VTA 205 includes the eyes, nose, and mouth of the human face 200. The first VTA 205 is defined as a set of coordinates (e.g., a range of coordinates), from the set of coordinates that define the display space of the visual presentation which in this case, the display space is the area of the computer monitor screen 210. In this example, visual prompt 215 is also included in the visual presentation in the form of a dotted line in a geometric shape circumscribing the first VTA 205.

The visual presentation shown in FIG. 2A is displayed for a user and, during this display, measurement data is collected from the user. This measurement data includes, among other things, eye tracking data indicating the user's gaze with respect to the first VTA 205. Based on the measurement data, a new, second VTA 220 is defined as shown in FIG. 2B.

As with the first VTA 205, the second VTA 220 may be defined based on a set of coordinates from the set of coordinates that define the display space of the visual presentation. In this case, the display space is the area of the computer monitor screen 210. The set of coordinates for the second VTA 220 are different than those used for the first VTA 205 because the former only covers the eyes and nose of the human face 200, while the latter covers the eyes, nose, and mouth of the human face 200. A visual prompt 225 is also included in the visual presentation in the form of a dotted line in a geometric shape circumscribing VTA 220. As an example of how this transformation may occur, consider a subject that is being trained to maintain a gaze on human eyes for a predetermined period of time. The first VTA 205 may be presented as the initial goal for this individual. If the subject maintains a gaze on the VTA 205 for the desired period of time (as determined by the measurement data), the size of the VTA can be reduced to further concentrate on the human's eyes as shown in the second VTA 220. Thus, the subject can be trained gradually over several iterations to reach the goal of eye contact. FIG. 2C provides an additional example where the VTA is narrowed even further in VTA 230 to focus on the eye portion of the human face depicted in the visual presentation. A visual prompt 235 is also included in the visual presentation in the form of a dotted line in a geometric shape circumscribing the VTA 230. FIG. 2D provides an additional example where the VTA 240 is the same as in FIG. 2C but the difficulty level for the user is increased by removal of the visual prompt.

It should be noted that the examples discussed above with reference to FIGS. 2A-2D are not limited to the types of faces displayed in the examples. For example, in other embodiments, the VTAs may display faces of animals and non-human imaginary faces as part of visual training. For example, a training strategy may be implemented whereby the user is gradually transitioned from non-human faces to human faces as part of the training.

In another example, reference is made to FIG. 1. As User 4's VGP Input 500 data shows User 4's gaze within the VTA for a certain period of time, the VTA would become smaller in size and different in shape for a certain period of time, then move to a different location for a certain period of time, requiring greater focus and representing a more challenging visual training. This training could further include CGE Parameters that include targeted physiological measurement data so that presentations of the VTAs (including variations in speed of presentation, frequency, location, and size), may also be determined in whole or in part based on this measurement data. This training may further include CGE Parameters that include targeted behavioral measurement data so that presentations of the VTAs (including variations in speed of presentation, frequency, location, and size), may also be determined in whole or in part based on this measurement data such as in training simulations in which the user is prompted to take an action that involves making a choice from among alternative choices presented to the user (which may be presented in the visual presentation), by using a computer mouse, game controller, or other device to make such selection which may be during presentation of the VTA This process could provide for training for targeted physiology and behavior during different forms of visual training that may involve challenging visual analysis and decision making tasks.

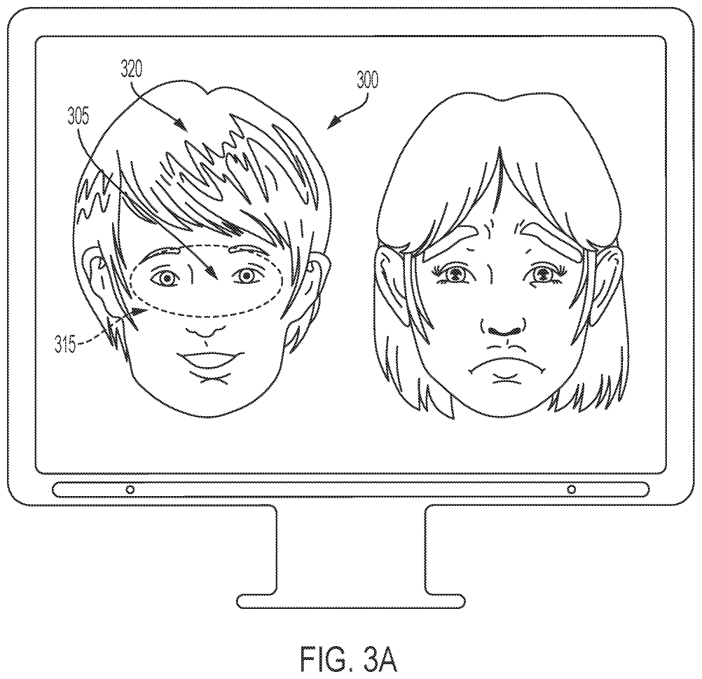

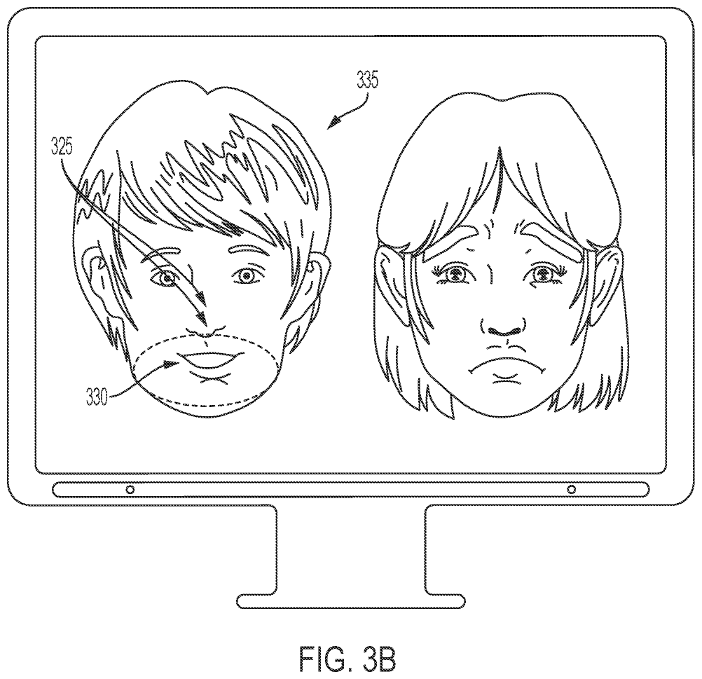

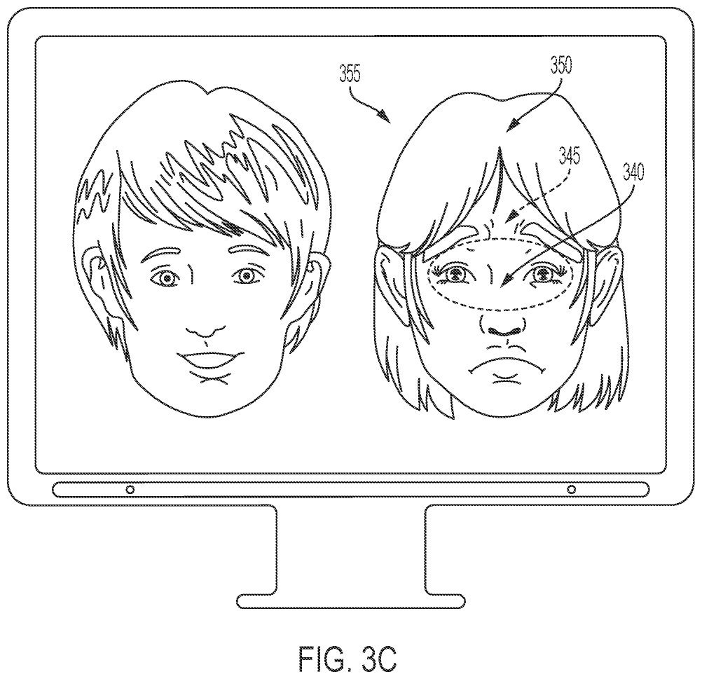

FIGS. 3A-3C illustrate an example of moving a VTA in response to collected measurement data, according to some embodiments. In FIG. 3A two game character faces may be presented in a visual presentation 300 such as a movie or video game in which a simulation of a social interaction with a group of individuals may be presented to the user. A first VTA 305 is located in the eye region of game character 320. A visual prompt 315 is also included in the visual presentation in the form of a dotted line in a geometric shape circumscribing the VTA 305. The visual presentation 300 shown in FIG. 3A is displayed for a user and, during this display, measurement data is collected from the user. This measurement data includes, among other things, eye tracking data indicating the user's gaze with respect to the first VTA 305.

Based on the measurement data, a new, second VTA 330 is defined in a different location as shown in FIG. 3B located over the mouth region of game character 320. A visual prompt 325 is also included in the visual presentation 335 in the form of a dotted line in a geometric shape circumscribing the VTA 330. The visual presentation 335 shown in FIG. 3B is displayed for a user and, during this display, measurement data is collected from the user.

This measurement data includes, among other things, eye tracking data indicating the user's gaze with respect to VTA 330.

Based on the measurement data, a new, third VTA 340 is defined in a different location as shown in FIG. 3C located over the eye region of game character 350. A visual prompt 345 is also included in the visual presentation 355 in the form of a dotted line in a geometric shape circumscribing the VTA 340. The visual presentation 355 shown in FIG. 3C is displayed for a user and, during this display, measurement data is collected from the user. This measurement data includes, among other things, eye tracking data indicating the user's gaze with respect to VTA 340.

As an example of how this transformation may occur, consider a subject that is being trained to make and/or maintain eye contact during interactions with multiple people. In this example, the training goal is for the subject to make and/or maintain eye contact with each game character for a predetermined period of time as the game character is speaking. The first VTA 305 may be presented as the initial goal for this individual. If the subject maintains a gaze on the VTA 305 for the desired period of time (as determined by the measurement data), the location of the VTA is then changed to VTA 330 to allow the subject an interval of visual focus other than human eye contact but still within a facial region (in this case the mouth region of game character 320), the subject is then prompted visually 345 to concentrate on a second human character's eyes as shown in the third VTA 340 as game character 350 is speaking. Thus, the subject can be trained iteratively to alternate his or her eye contact between different individuals in social interactions.

Applying VTAs in this way can be used for any training that requires sequential visual analysis by the trainee of a situation capable of being included in a visual presentation. This training could further include CGE Parameters that include targeted physiological measurement data so that presentations of the VTAs (including variations m speed of presentation, frequency, location, and size), may also be determined in whole or in part based on this measurement data. This training may further include CGE Parameters that include targeted behavioral measurement data so that presentations of the VTAs (including variations in speed of presentation, frequency, location, and size), may also be determined in whole or in part based on this measurement data such as in training simulations in which the user is prompted to take an action that involves making a choice from among alternative choices presented to the user (which may be presented in the visual presentation), by using a computer mouse, game controller, or other device to make such selection which may be during presentation of the VTA). This process could provide for training for targeted physiology and behavior during different forms of visual training that may involve challenging visual analysis and decision making tasks.

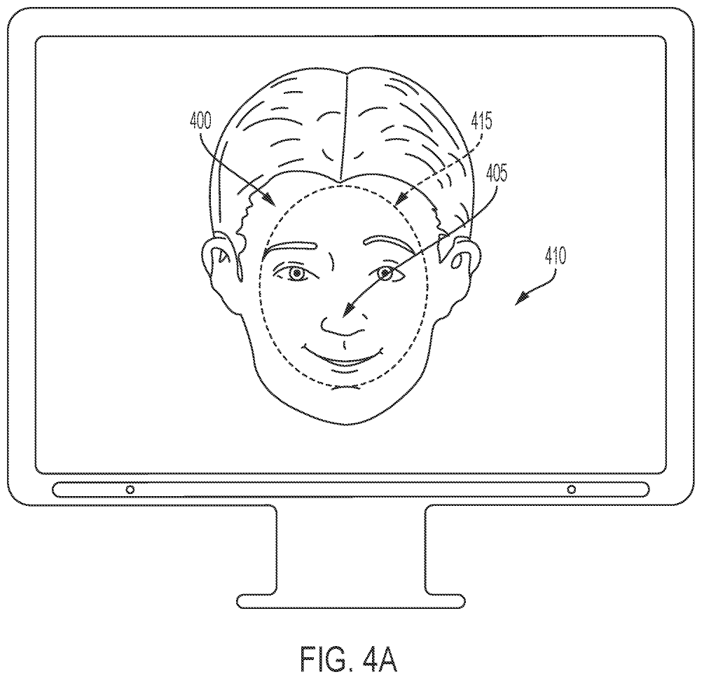

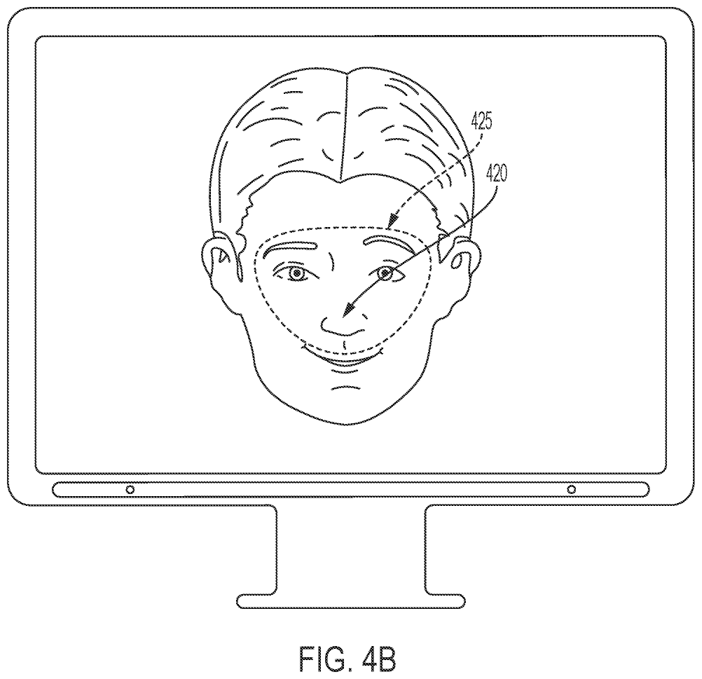

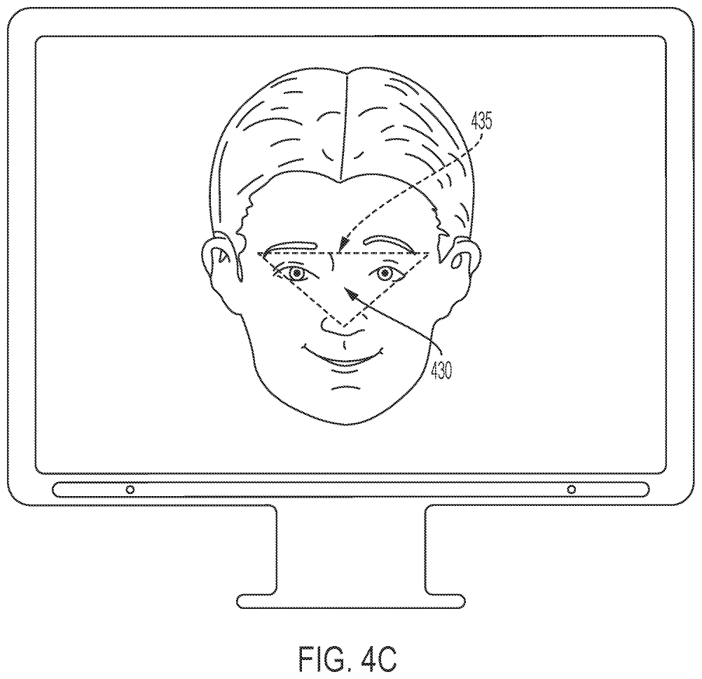

FIGS. 4A-4C illustrate an example of morphing a VTA in response to collected measurement data, according to some embodiments. Starting with FIG. 4A, a human face 400 is presented in a visual presentation such as a movie or video game which may be presented as a simulation of a social interaction with a single individual. A first VTA 405 is defined in the shape of a circle and includes the eyes, nose, and mouth of the human face 400. The first VTA 405 is defined as a set of coordinates (e.g., a range of coordinates), from the set of coordinates that define the display space of the visual presentation which in this case, the display space is the area of the computer monitor screen 410. In this example, visual prompt 415 is also included in the visual presentation in the form of a dotted line in a geometric shape of a circle circumscribing the first VTA 405.

The visual presentation shown in FIG. 4A is displayed for a user and, during this display, measurement data is collected from the user. This measurement data includes, among other things, eye tracking data indicating the user's gaze with respect to the first VTA 405. Based on the measurement data, a new, second VTA 420 is defined as shown in FIG. 4B.

As with the first VTA 405, the second VTA 420 may be defined based on a set of coordinates from the set of coordinates that define the display space of the visual presentation. In this case, the display space is the area of the computer monitor screen 410. The set of coordinates for the second VTA 420 are different than those used for the first VTA 405 because the former is shaped differently in the form of an inverted triangle with rounded corners and only covers the eyes and nose of the human face 400, while the latter is shaped in a circle that covers the eyes, nose, and mouth of the human face 400. A visual prompt 425 is also included in the visual presentation in the form of a dotted line the shape of an inverted triangle with rounded corners circumscribing VTA 420. As an example of how this transformation may occur, consider a subject that is being trained to maintain a gaze on human eyes for a predetermined period of time. The first VTA 405 may be presented as the initial goal for this individual. If the subject maintains a gaze on the VTA 405 for the desired period of time (as determined by the measurement data), the size and shape of the VTA can be changed to further concentrate on the human's eyes as shown in the second VTA 420. Thus, the subject can be trained gradually over several iterations to reach the goal of eye contact. FIG. 4C provides an additional example where the VTA is changed even further in shape and size to an inverted triangle VTA 430 to focus on the eye portion of the human face depicted in the visual presentation. A visual prompt 435 is also included in the visual presentation in the form of a dotted line in the shape of an inverted triangle circumscribing the VTA 430.

As an additional example of how this process could be applied, consider a training population that includes a spectrum disorder such as autism spectrum disorder. Because each individual's deficits can vary widely, training requires the ability to individualize the deployment of training strategies. The present example provides for a potential human eye contact training assessment by measuring the gaze on areas of human character's faces through deployment of differently shaped VTAs.

It should be noted that because the VTA may be defined by a set of coordinates from the set of coordinates that define the visual presentation, those set of coordinates may define multiple areas of the visual presentation and in some embodiments the VTA may comprise a plurality of non-contiguous areas (which may differ in size and shape) of the visual presentation and the associated prompts as visual indicators may be non-contiguous as well.

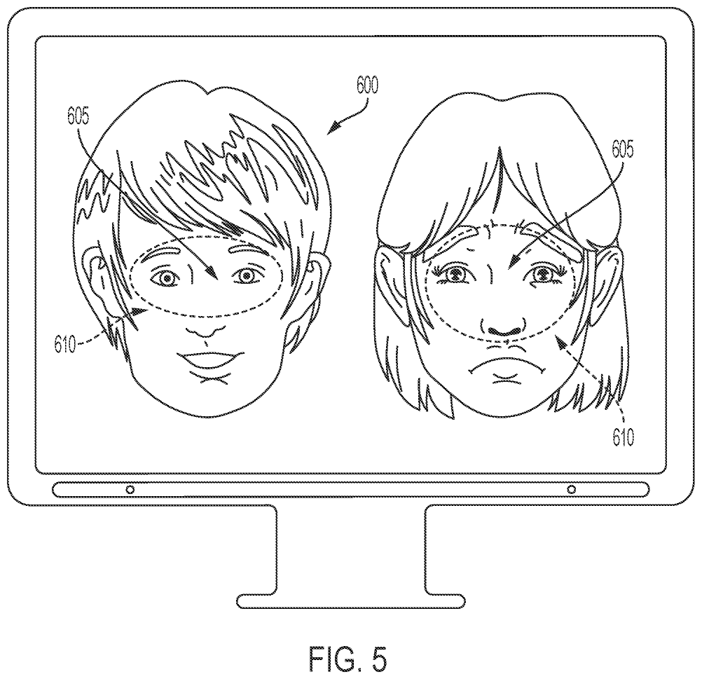

FIG. 5 provides an example where two human faces are presented to the user as part of a visual presentation. A first VTA 605 is defined as a set of coordinates (e.g., a range of coordinates), from the set of coordinates that define the display space of the visual presentation which in this case, the display space is the area of the computer monitor screen 600. The VTA comprises two non-contiguous areas of each of those faces which vary in size and shape from each other as shown in 605. A visual prompt 610 is also included in the visual presentation in the form of a dotted line in a geometric shape circumscribing the areas defined by VTA 605.

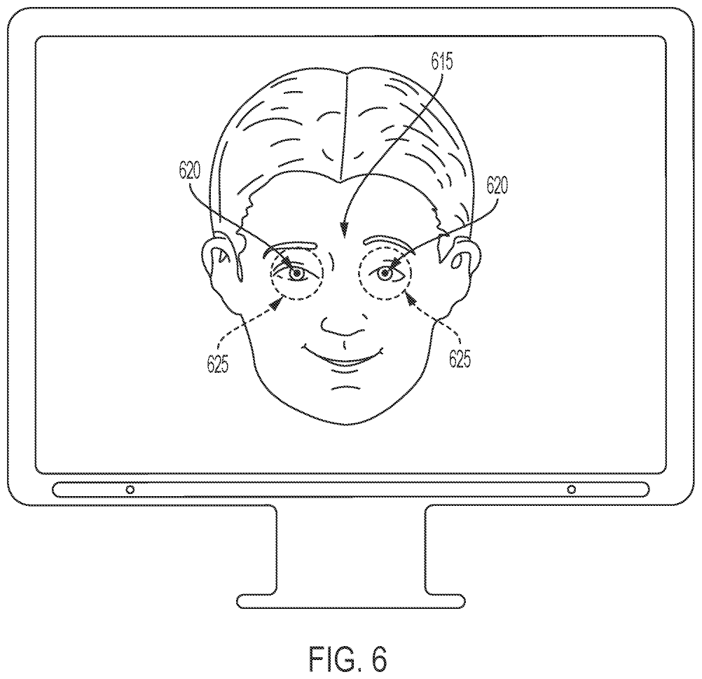

FIG. 6 provides an additional example where the VTA comprises two non-contiguous areas of the display space of the visual presentation. In this example a human face 615 is presented to the user as part of a visual presentation. The VTA comprises two non-contiguous areas of the face with each area covering each of the two eye regions of the face as shown in 620. A visual prompt 625 is also included in the visual presentation in the form of a dotted line in a geometric shape circumscribing the areas defined by VTA 620.

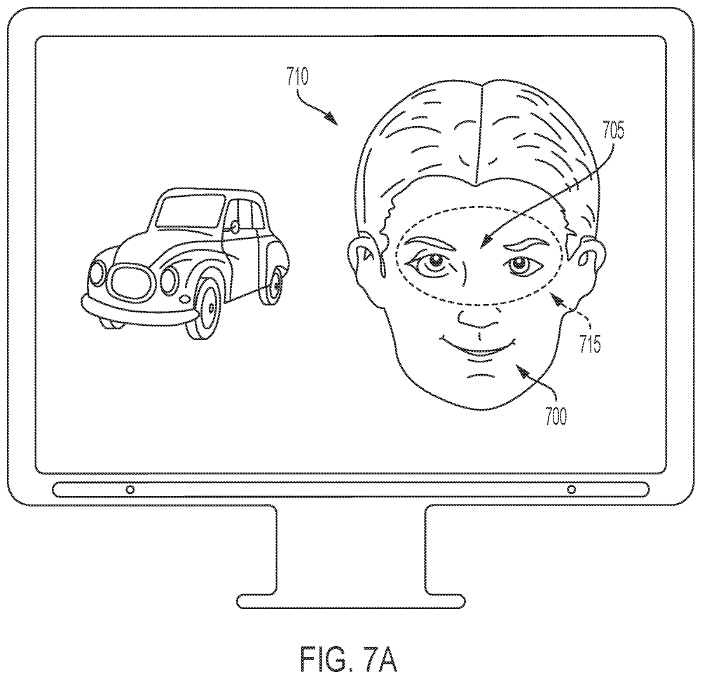

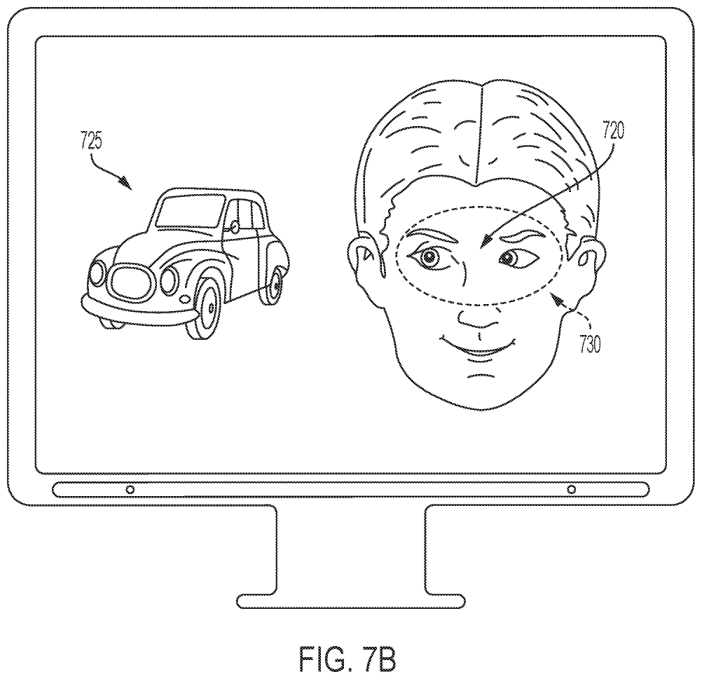

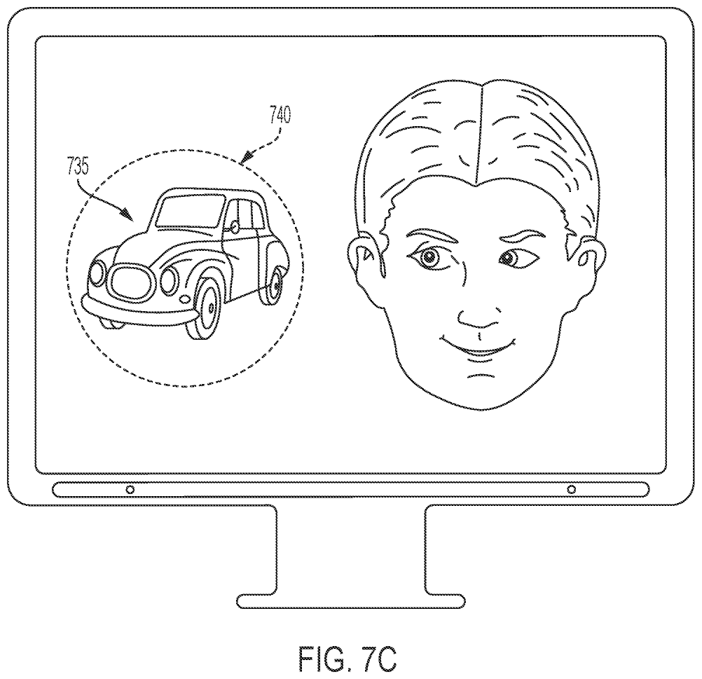

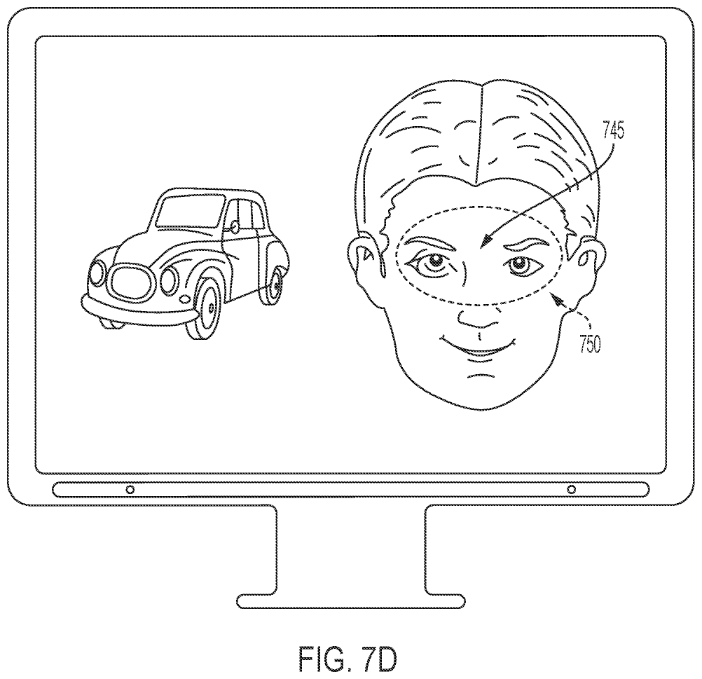

FIG. 7A through FIG. 7D provides another example of visual training which may involve a simulated joint attention exercise. Starting with FIG. 7A, a human face 700 is presented in a visual presentation such as a movie or video game which may be presented as a simulation of a social interaction with a single individual. A first VTA 705 is defined in the shape of an oval and includes the eyes of the human face 700. The first VTA 705 is defined as a set of coordinates (e.g., a range of coordinates), from the set of coordinates that define the display space of the visual presentation which in this case, the display space is the area of the computer monitor screen 710. In this example, visual prompt 715 is also included in the visual presentation in the form of a dotted line in a geometric shape of an oval circumscribing the first VTA 705.

The visual presentation shown in FIG. 7A is displayed for a user and during this display, measurement data is collected from the user. This measurement data includes, among other things, eye tracking data indicating the user's gaze with respect to the first VTA 705.

Based on the measurement data, a new, second VTA 720 is defined as shown in FIG. 7B in the shape of an oval that includes the eyes of the human face which appear to be looking at the object of interest 725 which in the visual presentation is a car. A visual prompt 730 is also included in the visual presentation in the form of a dotted line in a geometric shape of an oval circumscribing the second VTA 720.

The visual presentation shown in FIG. 7B is displayed for a user and during this display, measurement data is collected from the user. This measurement data includes, among other things, eye tracking data indicating the user's gaze with respect to the second VTA 720.

Based on the measurement data, a new, third VTA 735 is defined as shown in FIG. 7C in the shape of a circle that includes the object of interest car 725. A visual prompt 740 is also included in the visual presentation in the form of a dotted line in a geometric shape of a circle circumscribing the third VTA 735.

The visual presentation shown in FIG. 7C is displayed for a user and during this display, measurement data is collected from the user. This measurement data includes, among other things, eye tracking data indicating the user's gaze with respect to the third VTA 735.

Based on the measurement data, a new, fourth VTA 745 is defined as shown in FIG. 7D in the shape of an oval and includes the eyes of the human face 700. A visual prompt 750 is also included in the visual presentation in the form of a dotted line in a geometric shape of a circle circumscribing the third VTA 745.

The visual presentation shown in FIG. 7D is displayed for a user and during this display, measurement data is collected from the user. This measurement data includes, among other things, eye tracking data indicating the user's gaze with respect to VTA 745.

FIG. 8 illustrates an example of modifying a VTA to train user behavior for optimal collection of gaze data by an eye tracker during different forms of visual training. In this example, the visual presentation 825 includes the entire area of the computer monitor screen 800 and the VTA coordinates includes all of the coordinates of the computer monitor screen generating a VTA that is the same area as the computer monitor screen 800. The eye tracker 860 collects eye tracking measurement data indicating the user's gaze with the VTA and associates that data with the position of the user's 865 eyes in physical space in relation to the area in which the eye tracker 860 can capture complete and/or accurate eye tracking data (the “Eye Tracker Data Capture Field”) represented by the four brackets 830 positioned below the monitor screen 800 in the figure. Based on this measurement data, the system generates a next VTA that is associated with repositioning of the user's 865 eyes so that they fall within the Eye Tracker Data Capture Field. For example, the user's eye tracking measurement data in response to a first VTA indicates the user's eyes are positioned too far too the left in relation to Eye Tracker Data Capture Field. The system then generates a second VTA in 810 in the form of solid colored portion of the right side of the visual presentation 825. In 805 the user's eye tracking measurement data in response to the second VTA 810 indicates the user moved closer to the Eye Tracker Data Capture Field and a third VTA is generated decreasing the area of the solid colored portion of the right side of the visual presentation 825 from the previous VTA. In 800 the user's eye tracking measurement data in response to the third VTA 805 indicates the user's 865 eyes are within the Eye Tracker Data Capture Field and generates a fourth VTA that removes the area of the solid colored portion of the visual presentation 825. In this example, this process is also deployed where the user's 865 eyes are positioned too far too the right in relation to Eye Tracker Data Capture Field as illustrated in images 820 and 815.

FIG. 8 also illustrates a process in images 835 through 855 wherein the VTA presented includes a contiguous sold colored horizontal area and a sold colored vertical area of the visual presentation 825 associated with angle of the user's eyes in relation to Eye Tracker Data Capture Field.

FIG. 9 illustrates an additional process using eye tracking measurement data to generate VTAs to maintain the positioning of the user's eyes so that they fall within the Eye Tracker Data Capture Field. In this example, the visual presentation 910 includes the entire area of the computer monitor screen 900 and the VTA coordinates includes all of the coordinates of the computer monitor screen generating a VTA that is the same area as the computer monitor screen 900. The eye tracker 920 collects eye tracking measurement data indicating the user's gaze with the VTA and associates that data with the distance of the user's 925 eyes in physical space from the area in which the eye tracker can capture complete and/or accurate eye tracking data (i.e., the Eye Tracker Data Capture Field) which may be too close or too far from the eye tracker 920. Based on this measurement data, the system generates a next VTA that is associated with repositioning of the user's 925 eyes so that they fall within the Eye Tracker Data Capture Field. For example, the user's eye tracking measurement data in response to a first VTA indicates the user's eyes are positioned too close to the eye tracker 920 exceeding the boundary of the Eye Tracker Data Capture Field. The system then generates a second VTA in 905 in the form of blurred VTA which in this case includes the entire area of the visual presentation 910. In 910 the user's eye tracking measurement data in response to the second VTA 905 indicates the user 925 has repositioned user 925 eyes to an acceptable distance away from the eye tracker 920 so that user's 925 eyes are within the Eye Tracker Data Capture Field and generates a third VTA that removes the blurring of the visual presentation 910. In 915 the user's eye tracking measurement data in response to a first VTA indicates the user's eyes are positioned too far away from the eye tracker 920 exceeding the boundary of the Eye Tracker Data Capture Field. The system then generates a second VTA in 915 in the form of darkened VTA which in this case includes a darkening of the entire area of the visual presentation 910. In 910 the user's eye tracking measurement data in response to the second VTA 915 indicates the user 925 has repositioned user's 925 eyes to an acceptable distance away from the eye tracker 920 so that user's 925 eyes are within the Eye Tracker Data Capture Field and generates a third VTA that removes the darkening of the visual presentation 910.

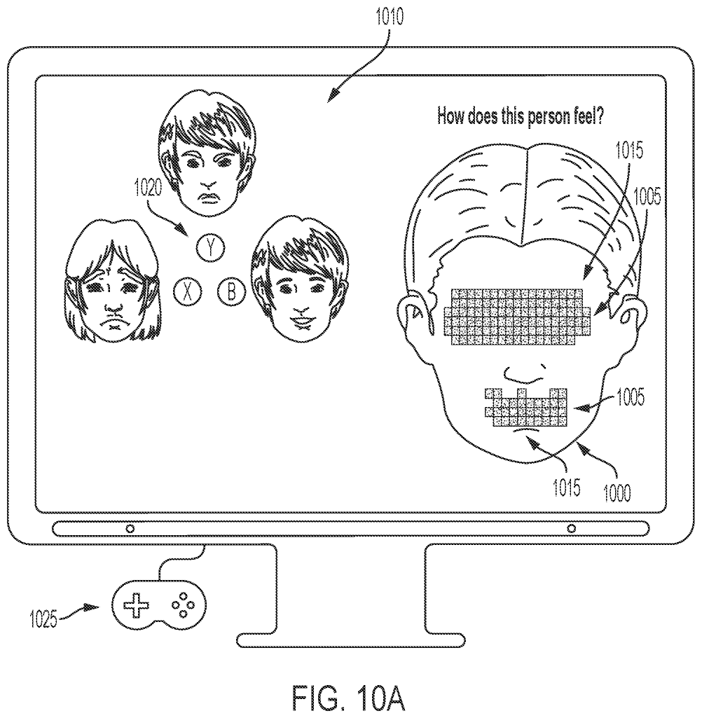

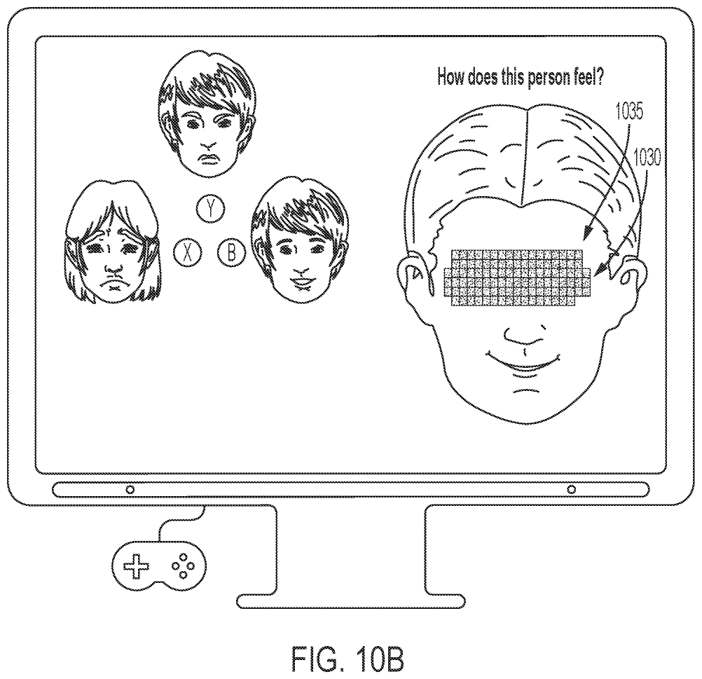

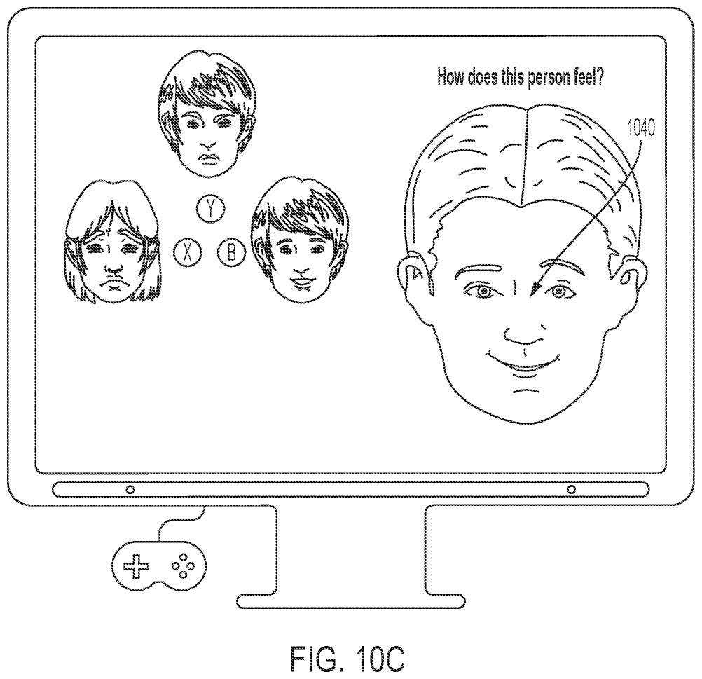

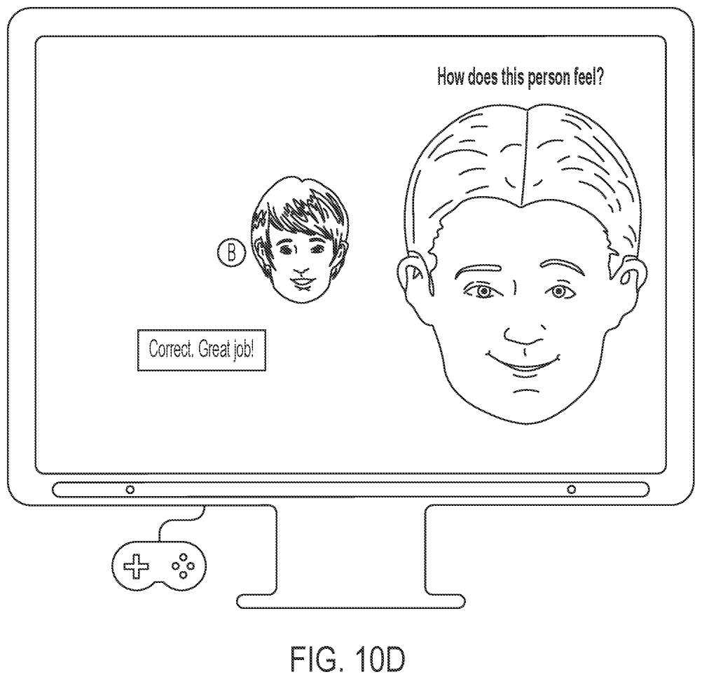

FIGS. 10A through 10D illustrates a process to train individuals including those with disabilities such as autism spectrum disorder to recognize the emotions of others using VTAs that are determined by both eye tracking measurement data and behavioral measurement data collected during a visual presentation.

Starting with FIG. 10A, a human face 1000 is presented in a visual presentation which in this case is a video game. The content of the visual presentation indicates that the object of the game is to match the emotion of the human face 1000 with a graphical depiction of the same emotion among a group of human faces 1020 presented as part of the visual presentation. The matching process is performed by selecting a letter depicted in the visual presentation that is associated visually with one of the representations of the human faces 1020 and which is also associated with a button on video game controller 1025 and the user pressing the game controller button associated with the selection.

A first VTA 1005 is defined by two non-contiguous areas of the human face 1000, one in the eye region of the face and the other in the mouth region. The first VTA 1005 is defined as a set of coordinates (e.g., a range of coordinates), from the set of coordinates that define the display space of the visual presentation which in this case, the display space is the area of the computer monitor screen 1010. In this example, a visual prompt 1015 is also included in the visual presentation in the form of a blurring of the first VTA 1005.

The visual presentation shown in FIG. 10A is displayed for a user and during this display, measurement data is collected from the user. This measurement data includes, among other things, eye tracking data indicating the user's gaze with respect to the first VTA 1005 and behavioral measurement data in the form of a press of one of the game controller buttons.

Based on the eye tracking and behavioral measurement data collected during the visual presentation of the first VTA 1005 a new, second VTA 1030 is defined as shown in FIG. 10B as the eye region of the human face 1000. In this example, a visual prompt 1035 is also included in the visual presentation in the form of a blurring of the second VTA 1030.

The visual presentation shown in FIG. 10B is displayed for a user and during this display, measurement data is collected from the user. This measurement data includes, among other things, eye tracking data indicating the user's gaze with respect to the second VTA 1030 and behavioral measurement data in the form of a press of one of the game controller buttons.

Based on the eye tracking and behavioral measurement data collected during the visual presentation of the second VTA 1030, a new, third VTA 1040 is defined as the eye, nose and mouth region of the of the human face 1000 with no visual prompt as shown in FIG. 10C.

The visual presentation shown in FIG. 10C is displayed for a user and during this display, measurement data is collected from the user. This measurement data includes, among other things, eye tracking data indicating the user's gaze with respect to the second VTA 1040 and behavioral measurement data in the form of a press of one of the game controller buttons.

Based on the eye tracking and behavioral measurement data collected during the visual presentation of the third VTA 1040, no VTA is presented to the user during the next visual presentation as the user successfully matched the emotion as shown in FIG. 10D.

This example demonstrates a process in which the training goal of recognizing the emotions of others can be deployed by teaching the user, iteratively, to visually scan certain areas of the face to collect the visual information necessary in order to ascertain the emotion presented.

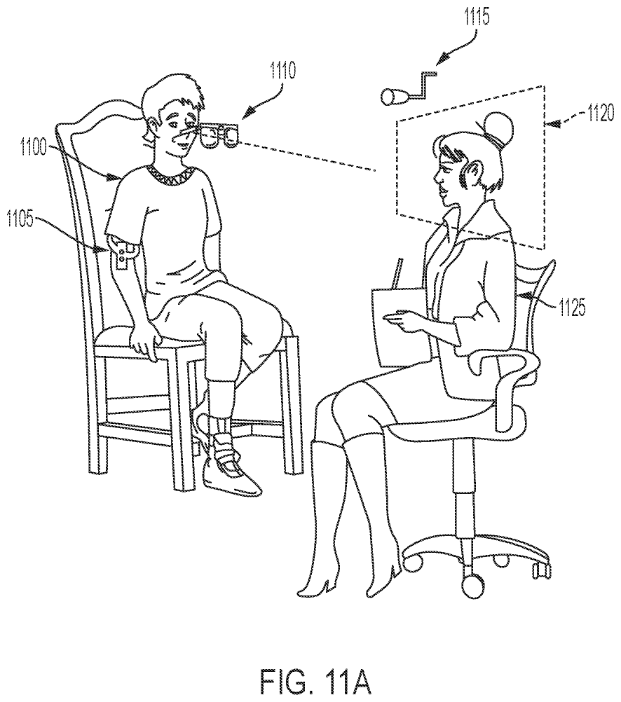

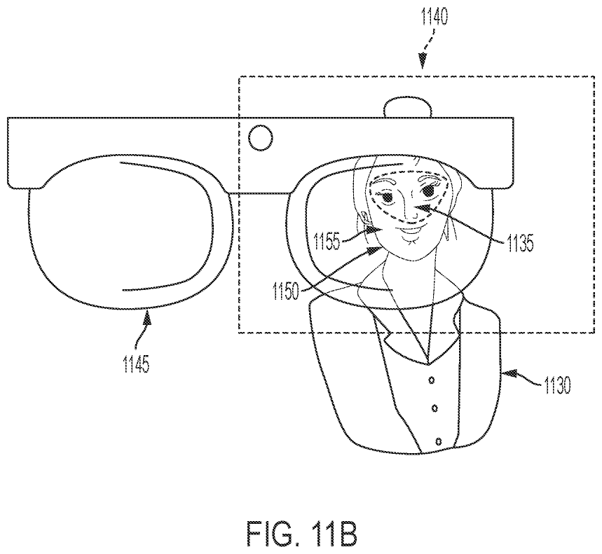

FIGS. 11A and 11B illustrates a process to train individuals including those with disabilities such as autism spectrum disorder to make and/or maintain eye contact in real world interactions based on eye tracking data collected during a visual presentation in which physiological and/or behavioral measurement data may also be collected during such visual presentation and may also be used.

Starting with FIG. 11A, a subject 1100 is in the same physical space as another individual which in this example is a Service Provider 1125 in the form of a therapist. The subject 1100 is wearing wireless real world eye tracking glasses 1110 capable of presenting graphical visual representations to the user while the user views the real world environment. Subject 1100 is also wearing a wireless physiological measuring device 1105 which in this example measures the subject's heart rate. The physical space also includes a motion capture device 1115 that can capture subject 1100 behavioral data which may include physical movements during interactions with Service Provider 1125.

Service Provider 1125 engages in a visual presentation, which may be in the form of a social interaction role play, presented to subject 1100 in which the coordinates of the visual presentation may be defined by subject 1100 viewing area 1120.

FIG. 11B shows the subject 1100 viewing perspective wireless real world eye tracking glasses 1145 are used by the subject 1100 to view a viewing area 1140 in the real world environment that includes the Service Provider 1130. The visual presentation area 1135 (which may be defined based on the viewing area 1140) is shown from the viewing perspective of the subject 1100.

A first VTA 1135 is presented during the visual presentation that includes the eyes and nose on the face 1150 of Service Provider 1130. The first VTA 1135 is defined as a set of coordinates (e.g., a range of coordinates), from the set of coordinates that define the subject 1100 viewing area 1140. In this example, visual prompt 1155 is also included in the visual presentation in the form of a dotted line in a geometric shape circumscribing the first VTA 1135.

The visual presentation shown in FIG. 11B is displayed for subject 1100 and during this display, measurement data is collected from the user. This measurement data includes, among other things, eye tracking data indicating the user's gaze with respect to the first VTA 1135. Based on the measurement data, anew, second VTA is defined and presented to subject 1100. As described in other embodiments, the second VTA presented may vary in size, shape and form and based on any other CGE Parameters and may or may not include a visual prompt. In this way subject 1100 can be presented with VTAs over time which vary in difficulty which may provide for iterative training to make and/or maintain real world eye contact.

The process described in this example may also include use of physiological measurement data collected during the presentation of the first VTA, which in this case could be heart rate measurement data using physiological measuring device 1105, to determine the second VTA.

The process described in this example may also include use of behavioral measurement data (in addition to eye tracking data) collected during the presentation of the VTA, which in this case could include certain of subject 1100 body movements during presentation of the VTA using motion capture device 1115, to determine the second VTA. Additionally, the process described in this example may also include use of both physiological measurement data collected during the presentation of the VTA (which in this case could be heart rate measurement data using physiological measuring device 1105) and behavioral measurement data (in addition to eye tracking data) collected during the presentation of the VTA (which in this case could include certain of subject 1100 body movements during presentation of the VTA using motion capture device 1115) to determine the second VTA.

Use of real world eye tracking measurement data, together with physiological and behavior measurement data, collected during presentation of each VTA to determine each subsequent VTA in a visual presentation may provide for a process that can achieve better outcomes in meeting training goals for improved social skills by being able to deliver more challenging VTAs gradually without overloading the emotional and mental state of the individual being trained. This is especially important to achieve training goals with respect to individuals with disabilities such as autism spectrum disorder.

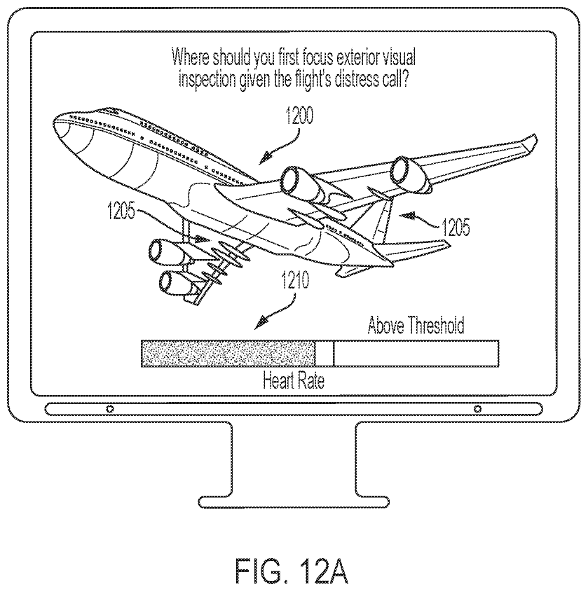

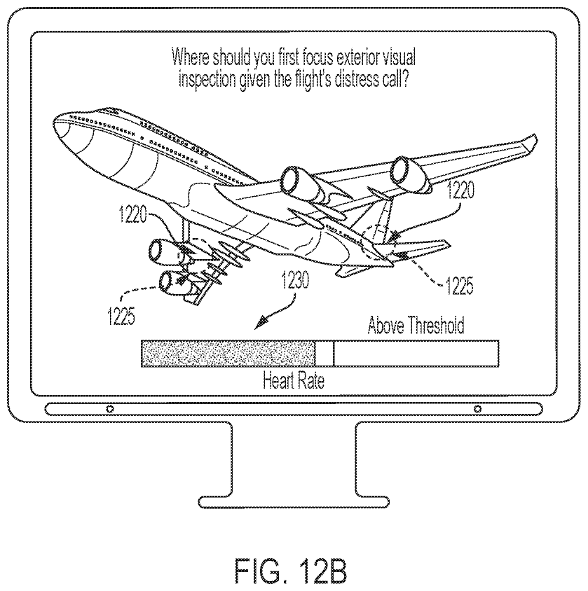

FIG. 12A and FIG. 12B provide another example of how this process can be used to train for critical skills as part of training simulations. In FIG. 12 A the user is wearing a wireless physiological measuring device which in this example measures the subject's heart rate. A graphical representation of the acceptable heart rate threshold 1210 is presented as part of the visual presentation. The user in this example is an airplane service technician and the visual presentation presents an airplane 1200 that the user is aware is in mechanical distress.

A first VTA 1205 is defined by two non-contiguous areas of the airplane 1200. The first VTA 1205 is defined as a set of coordinates (e.g., a range of coordinates), from the set of coordinates that define the display space of the visual presentation which in this case, the display space is the area of the computer monitor screen. In this example, a visual prompt is not included in the visual presentation.

The visual presentation shown in FIG. 12A is displayed for a user and during this display, measurement data is collected from the user. This measurement data includes, among other things, eye tracking data indicating the user's gaze with respect to the first VTA 1205 and physiological measurement data collected during display of the first VTA 1205.

Based on the eye tracking and physiological measurement data collected during the visual presentation of the first VTA 1205 a new, second VTA 1220 is defined as shown in FIG. 12B as the same two regions of the airplane 1200 as in the first VTA but in this instance a visual prompt 1225 is also included in the visual presentation in the form of a dotted line in a geometric shape circumscribing the second VTA 1220.

The visual presentation shown in FIG. 12B is displayed for a user and during this display, measurement data is collected from the user. This measurement data includes, among other things, eye tracking data indicating the user's gaze with respect to the second VTA 1220 and physiological measurement data collected during display of the second VTA 1220. Once again a graphical representation of the acceptable heart rate threshold 1230 is presented as part of the visual presentation.

The training sequences may be repeated with the training goal of successful visual inspection without the use of any prompts and/or maintenance of a desirable physiological state during visual inspection which may include when inspection time is limited due to safety concerns with significant consequences to human life.

This example indicates how the system can be used to foster visual training of sensitive machines that involve public safety while also training the user to maintain a calm mental state by training the user to be mindful of the user's physiological response which in this example was the user's heart rate.

In another similar example, the system is used to conduct visual training while collecting physiological and behavioral measurement data to train for repair of complex machines under time-sensitive conditions.

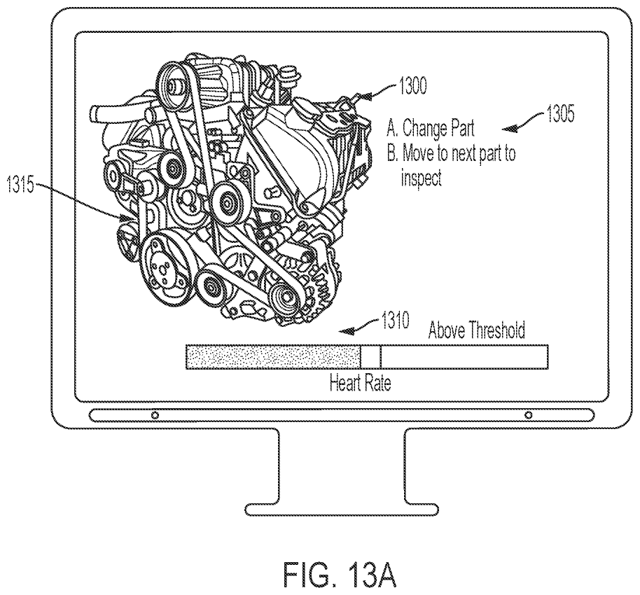

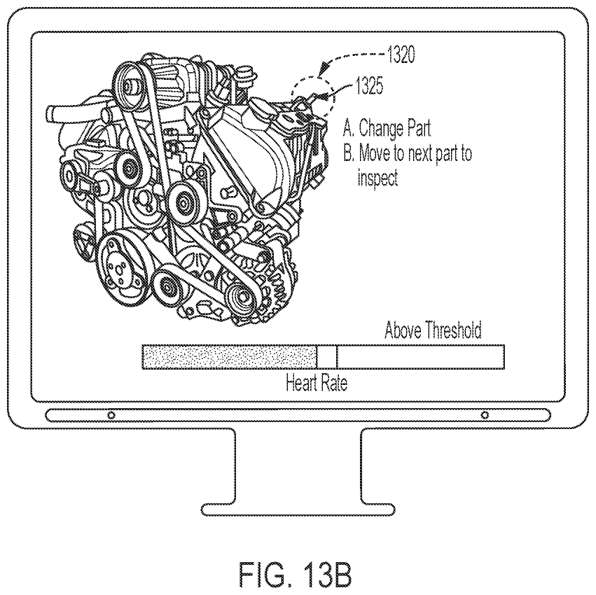

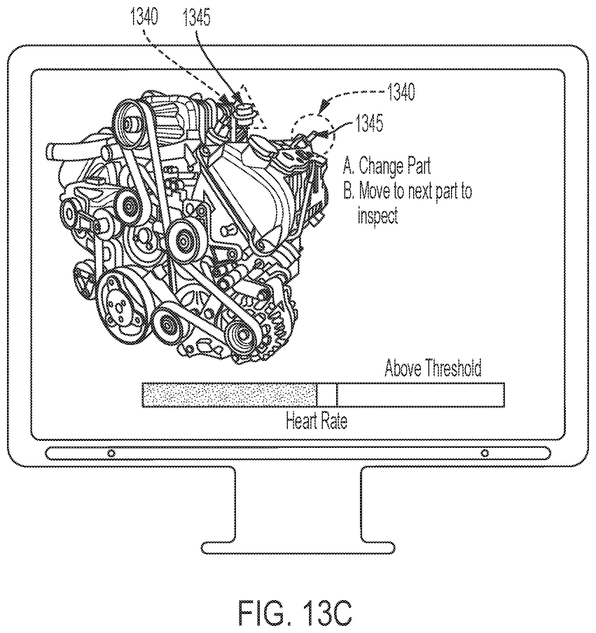

FIG. 13A through FIG. 13C provide another example of how this process can be used to train for critical skills as part of training simulations. In FIG. 13A, the user is wearing a wireless physiological measuring device which in this example measures the subject's heart rate. A graphical representation of the acceptable heart rate threshold 1310 is presented as part of the visual presentation. The users of this training process may include machine service technicians that perform work on sensitive and potentially dangerous machines. The visual presentation in this example includes presentation of an engine 1315. The user is also provided with a keyboard with which to input behavioral measurements during presentation of VTAs.

A first VTA 1300 is defined by an areas of the engine displayed in the visual presentation. The first VTA 1300 is defined as a set of coordinates (e.g., a range of coordinates), from the set of coordinates that define the display space of the visual presentation which in this case, the display space is the area of the computer monitor screen. In this example, a visual prompt is not included in the visual presentation. The visual presentation also includes a list of possible actions 1305 in text format which the user may select from by using the keyboard.

The visual presentation shown in FIG. 13A is displayed for a user and during this display, measurement data is collected from the user. This measurement data includes, among other things, eye tracking data indicating the user's gaze with respect to the first VTA 1300, physiological measurement data collected during display of the first VTA 1300 and behavioral measurement data in the form of keyboard entries by the user.

Based on the eye tracking, physiological and behavioral measurement data collected during the visual presentation of the first VTA 1300 a new, second VTA 1325 is defined as shown in FIG. 13B as the same regions of the engine 1315 as in the first VTA but in this instance a visual prompt 1320 is also included in the visual presentation in the form of a dotted line in a geometric shape circumscribing the second VTA 1325.

The visual presentation shown in FIG. 13B is displayed for a user and during this display, measurement data is collected from the user. This measurement data includes, among other things, eye tracking data indicating the user's gaze with respect to the second VTA 1325, and physiological and behavioral measurement data collected during display of the second VTA 1325.

Based on the eye tracking, physiological and behavioral measurement data collected during the visual presentation of the second VTA 1325 a new, third VTA 1345 is defined as shown in FIG. 13C as two non-contiguous regions of the engine 1315. A visual prompt 1340 is also included in the visual presentation in the form of a dotted line in a geometric shape circumscribing the third VTA 1345.

The visual presentation shown in FIG. 13C is displayed for a user and during this display, measurement data is collected from the user. This measurement data includes, among other things, eye tracking data indicating the user's gaze with respect to the third VTA 1345, physiological measurement data collected during display of the third VTA 1345 and behavioral measurement data in the form of keyboard entries by the user.

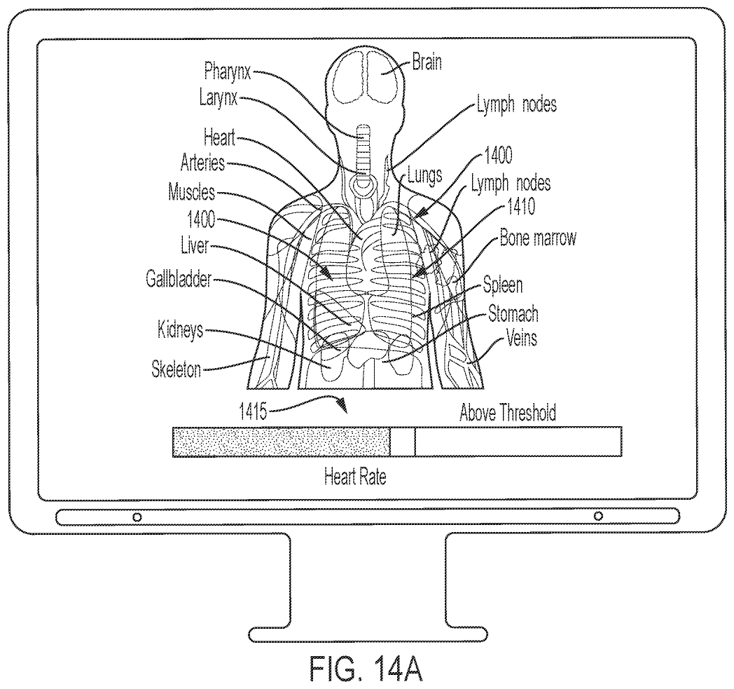

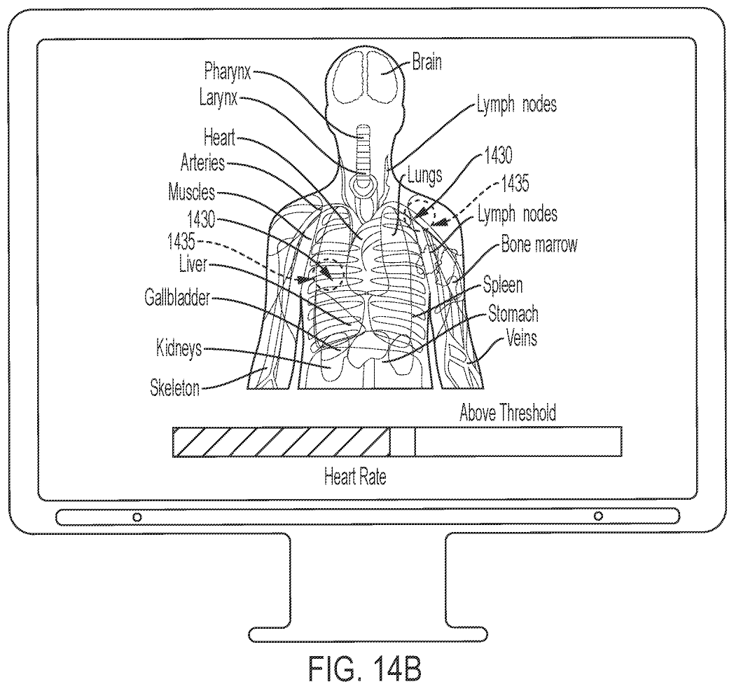

FIG. 14A and FIG. 14B illustrate how the process can be used to help train emergency medical personnel as part of training simulations. In FIG. 14A the user is wearing a wireless physiological measuring device which in this example measures the subject's heart rate. A graphical representation of the acceptable heart rate threshold 1415 is presented as part of the visual presentation. The user in this example may include an emergency medical personnel trainee and the visual presentation includes a presentation of an anatomical representation of the human body 1410.

A first VTA 1400 is defined by two non-contiguous areas of the body. The first VTA 1400 is defined as a set of coordinates (e.g., a range of coordinates), from the set of coordinates that define the display space of the visual presentation which in this case, the display space is the area of the computer monitor screen. In this example, a visual prompt is not included in the visual presentation.

The visual presentation shown in FIG. 14A is displayed for a user and during this display, measurement data is collected from the user. This measurement data includes, among other things, eye tracking data indicating the user's gaze with respect to the first VTA 1400 and physiological measurement data collected during display of the first VTA 1400.

Based on the eye tracking and physiological measurement data collected during the visual presentation of the first VTA 1400 a new, second VTA 1430 is defined as shown in FIG. 14B as the same two regions of the human body 1410 as in the first VTA but in this instance a visual prompt 1435 is also included in the visual presentation in the form of a dotted line in a geometric shape circumscribing the second VTA 1430.

The visual presentation shown in FIG. 14B is displayed for a user and during this display, measurement data is collected from the user. This measurement data includes, among other things, eye tracking data indicating the user's gaze with respect to the second VTA 1430 and physiological measurement data collected during display of the second VTA 1430.

The training sequences may be repeated with the training goal of successful visual inspection of the human body during simulated rendering of medical assistance without the use of any prompts and/or maintenance of a desirable physiological state during such activity which may include when time is limited due to safety concerns with significant consequences to human life.

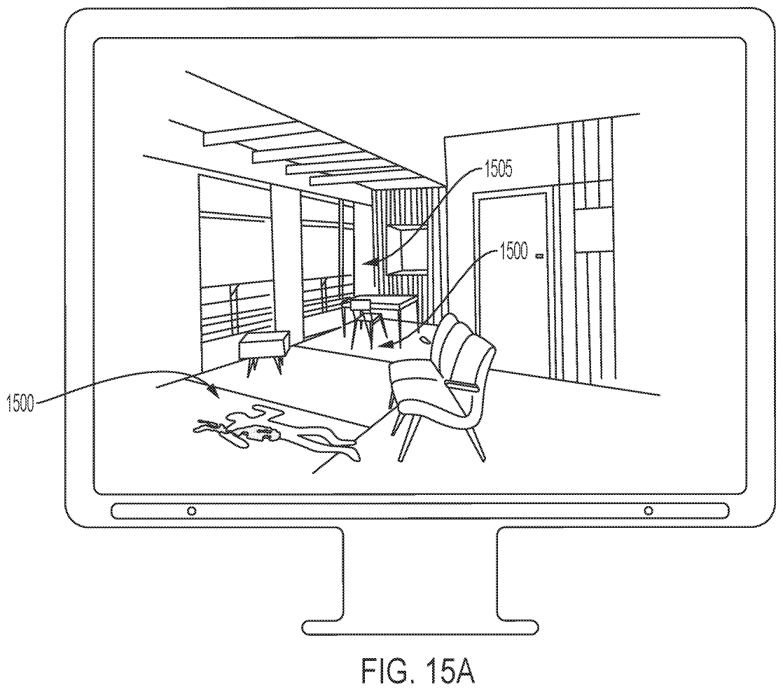

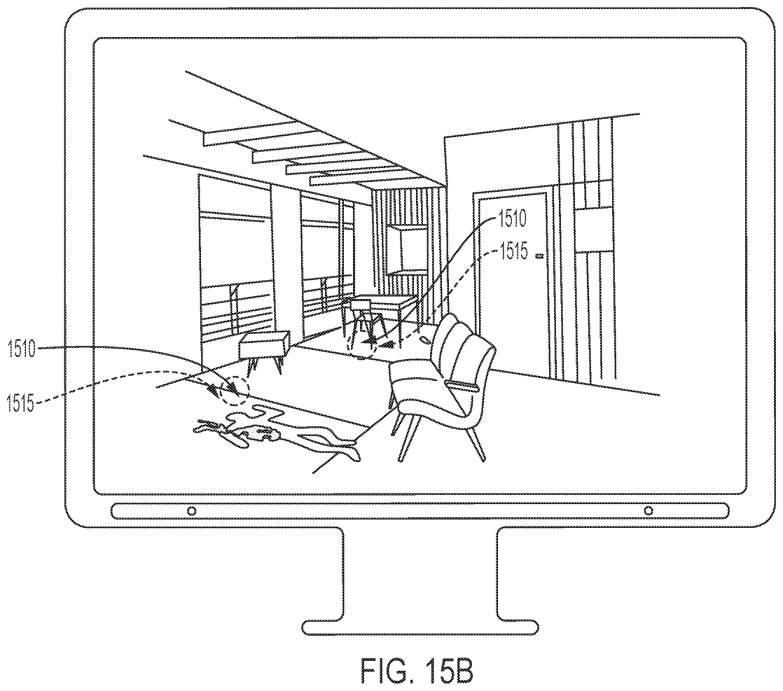

FIG. 15A and FIG. 15B illustrate how the process can be used to help train forensic law enforcement personnel as part of training simulations. In FIG. 15A the visual presentation includes a presentation of a crime scene 1505.

A first VTA 1500 is defined by two non-contiguous areas of the crime scene 1505. The first VTA 1500 is defined as a set of coordinates (e.g., a range of coordinates), from the set of coordinates that define the display space of the visual presentation which in this case, the display space is the area of the computer monitor screen. In this example, a visual prompt is not included in the visual presentation.

The visual presentation shown in FIG. 15A is displayed for a user and during this display, measurement data is collected from the user. This measurement data includes, among other things, eye tracking data indicating the user's gaze with respect to the first VTA 1500.

Based on the eye tracking and physiological measurement data collected during the visual presentation of the first VTA 1500 a new, second VTA 1510 is defined as shown in FIG. 15B as the same two regions of the crime scene 1505 as in the first VTA but in this instance a visual prompt 1515 is also included in the visual presentation in the form of a dotted line in a geometric shape circumscribing the second VTA 1510.

The visual presentation shown in FIG. 15B is displayed for a user and during this display, measurement data is collected from the user. This measurement data includes, among other things, eye tracking data indicating the user's gaze with respect to the second VTA 1510.

The training sequences may be repeated with the training goal of successful visual inspection of crime scenes during the conducting of simulated forensic investigations without the use of any prompts.

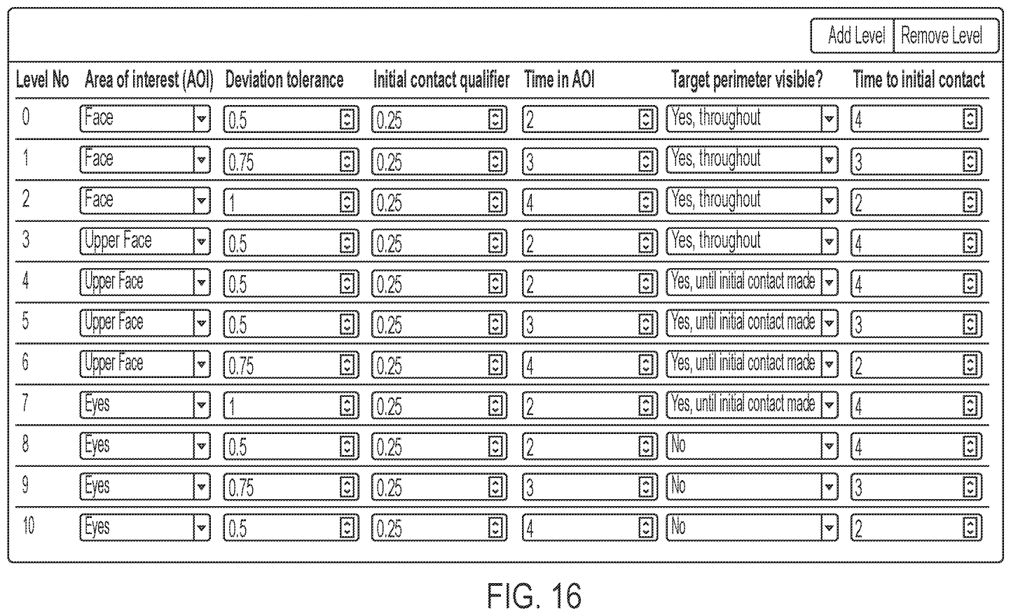

FIG. 16 illustrates an example GUI that may be used by Service Provider for entering some of the CGE Parameters used by the CEGS for a visual training sequence that trains a user to view the eyes of a human face. Note that the Service Provider sets the values such that difficulty of the training increases as the user proceeds through levels. For example, at levels 0-2, the user only needs to view the face generally; however, as the level increases, the deviation tolerance is gradually decreased and time in area of interest (AOI) is gradually increased to make the scenario more difficult. Similarly, at levels 3-6, the user is required to view the upper portion of the human face with the deviation tolerance and time in AOI adjusted in a manner similar to that described above. Finally, at levels 7-10, the user is required to view the eyes of the human face with similar adjustments to deviation tolerance and time in AOI adjusted as the level increases. It should be further noted that other parameters such as whether a prompt is presented (“Target perimeter visible?”) and the time to initial contact are also provided with values that make the training scenarios increasingly difficult for the user. As shown in the example of FIG. 16, the GUI includes two buttons (labeled “Add Level” and “Remove Level”) that allow the service provider to add or remove levels from the training exercise. In this way, the service provider can create custom sequences tailored to the training goals for the individual user.

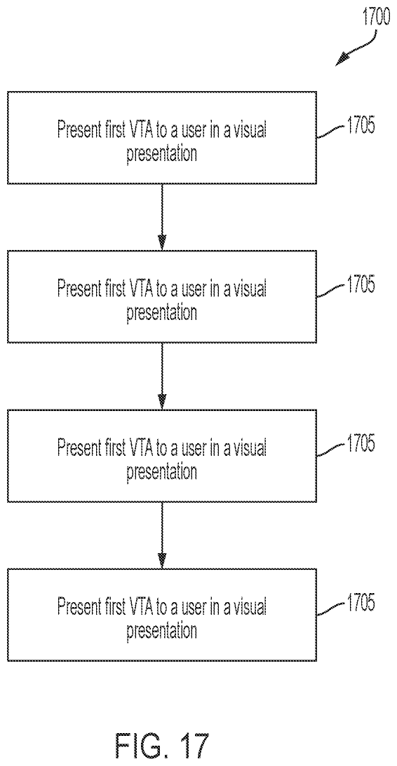

FIG. 17 illustrates a computer-implemented method 1700 for adaptive behavioral training, according to some embodiments. Starting at step 1705, a first VTA is presented to a user within a visual presentation. The first VTA may be defined, for example, based on one or more training goals. For example, for a user being trained to maintain eye contact, a human face may be displayed in the visual presentation. Then, the first VTA may be defined as an area of the human face that includes the eyes (and possibly other elements of the face). The visual presentation has a defined coordinate space within which the first visual training is defined. In some embodiments, the set of coordinates defining the first VTA may be entered by the person(s) administering the test (referred to herein as the “Service Provider”). For example, in one embodiment, the Service Provider may specify a range of coordinate values specifying where in the visual presentation the VTA should be located. In other embodiments, the computing system implementing the method 700 may automatically determine the set of coordinates based on a specified training goal. For example, in one embodiment, the Service Provider specifies the goal (e.g., “maintain eye contact”) and the computing system uses predetermined rules to determine the area, and by extension, the coordinates. In other embodiments, the testing administer is able to draw the VTA in a GUI and the computing system uses this information to derive the set of coordinates.

In some embodiments, the method 1700 further includes prompting the user to view the first VTA, the user may be prompted with an auditory prompt, a visual prompt, or a prompt that includes auditory and visual aspects. The visual prompt may take the form, for example, of a visual indicator of the training area. This visual indicator may be, for example, a graphical depiction of the perimeter of the VTA, brightening or darkening the area of the VTA, blurring of the VTA, or a graphic screen overlay of VTA comprised of different graphical elements. In one embodiment, the visual indicator is a geometric shape circumscribing, or otherwise depicting the boundary of, the first VTA.

Continuing with reference to FIG. 17, step 1710, measurement data is collected while the first VTA is presented to the user. This measurement data may include various types of measurement related to how the user is physically reacting to presentation of the visual presentation. For example, in some embodiments, the measurement data comprises eye tracking measurement data indicating the user's gaze with respect to the first VTA. The term “eye tracking measurement data” refers to coordinates indicating the user's gaze with respect to a VTA. Thus, eye tracking measurement data is derived by comparing collected eye tracking measurements with the set of coordinates defining the first VTA. Other examples of measurement data that may be collected at step 1710 include physiological measurement data indicating one or more user physiological responses (e.g., pulse rate) during presentation of the first VTA, and behavioral measurement data indicating one or more user behavioral responses (e.g., head positioning data, head stability data, etc.) during presentation of the first VTA.

It should be noted that the user may not be viewing the VTA at all in some instances. As described above, the VTA is defined by a set of coordinate values. One or more eye tracking devices collect data indicating the coordinates of the user's gaze. If the coordinates of the user's gaze fall within the coordinates of the VTA, the tracking measurement data will indicate that the user is viewing the VTA. Conversely, the coordinates of the user's gaze are outside of that area, the eye tracking measurement data will indicate that the user is not viewing the VTA. In some embodiments, a deviation tolerance may be associated with the eye tracking measurement data. This deviation tolerance indicates how long a user must consistently view the VTA. For example, if the deviation tolerance is set to be 0.10 seconds, the user's gaze views the VTA but moves out of the VTA for 0.01 seconds, the eye tracking measurement data will indicate that the user viewed the VTA. Alternatively, if the user's gaze moves out of the VTA for 0.5 seconds, the eye tracking measurement data would indicate that the user did not view the VTA.

In some embodiments, the eye tracking measurement data indicates that the user is viewing the VTA if coordinates associated with the user's gaze are within the first set of coordinates defining the first training area. The eye tracking measurement data may further indicate the duration of time during which the eye tracking measurement data indicates that the user's gaze is within the first VTA. In some embodiments, the duration of time indicates cumulative value, whereas in other embodiments it provides an indication of how long a user continuously views the first VTA. This time interval may be used as a “qualifier” for determining what viewing of the VTA should be considered “viewing” for the purposes of training. For example, the Service Provider may indicate that the user must continuously view the training area for at least 0.25 seconds in order to qualify as having viewed the first VTA. Any viewing that does not meet these criteria would then be ignored.

Returning to FIG. 17, at step 1715, a new, second VTA is selected based on the measurement data. As with the first VTA, the second visual training is defined by a set of coordinates. Thus, step 1715 can be understood as transforming the first set of the coordinates to the second set of coordinates based on the collected measurement data. For example, the second set of coordinates can move the first VTA to a second training area. Alternatively (or additionally), the second set of coordinates can expand the VTA, contract the VTA, or morph the shape of the VTA. The various transformations of the VTA are further illustrated in FIGS. 2A-6C. Finally, at step 1720, the second VTA is presented to the user in the visual presentation.

FIG. 8 provides an example of an interface for setting CGE Parameters, according to some embodiments. For example, a Service Provider conducts an assessment and/or performs a form of therapy and/or training for the user. The Service Provider from time to time inputs and/or transmits CGE Parameters to the Controller with respect to the user based in whole or in part on the Service Provider's interaction with the user including based on the Service Provider's assessment of the user and/or the behavior of the user in response to therapy and/or training conducted by the Service Provider.

This visual training technology described here may have applications in a broad variety of fields. Commercial applications include instances where it is important to train for visual attention (including sequential visual focus) which could be included as part of training simulations for delivering emergency medical treatment (and other emergency response situations), troubleshooting and repair of complex machines and technology, any other situations where efficient visual analysis is a key component to performance (such as surgeries, athletic competitions, interrogations, crime scene investigation by detectives, antique furniture/art appraisal, and construction work).

Therapeutic applications include using the technology as part of social skills training for individuals with different medical and/or emotional conditions that result in impaired eye contact during social situations. This may include broader applications for purely social challenges such as inclusion as a broader solution for techniques to overcome shyness. It may further include helping people visually scan complex social scenes such as group meetings or parties in order to extract valuable information about the meeting environment and its participants.

Further applications include: diagnostic applications, such as a method to diagnose medical disorders or illnesses, including where patterns in users' CGE data (including singular or multiple physiologic data streams) can be used as basis or support for diagnosis; educational applications such as a method of conveying information and/or methods of information processing, or otherwise facilitating learning; assessment applications such as a method for assessing a user's current state in regards to any of the above applications (e.g. current policing skill in certain scenarios, current ability to make eye contact, current severity of certain disorders, or current amount of information known); and ancillary applications such as part of any application whose goal is to improve behavioral, physiological, and/or mental performance of some sort and/or train, educate, or assess.

Further applications exist where visual training is combined with physiology. This includes all of the above described applications (and others) where engaging in visual analysis while maintaining a targeted physiologic and mental state is important. The system provides for the ability to alter the CGE in response to physiology in order to induce a wide variety of targeted physiological states. These could include altering the CGE (including complex VTA patterns over time in potentially rapid sequence) with the goal of increasing the user's cognitive load so as to provide for training simulations under stressful situations where maintaining a calm state, mental focus, and required visual analysis (including sequential visual analysis) is critical to a successful outcome. Machine learning and artificial intelligence could be used to develop the best VTA patterns to deploy (and other CGE elements) on an individualized basis so as to most efficiently achieve the desired outcome. This could incorporate VTA pattern banks for testing and refinement over time across users globally.

All of the above described applications could be further configured such that multiple users simultaneously engage in a single CGE on a single machine, multiple users simultaneously engage in a single CGE on multiple machines, or multiple users simultaneously engage in multiple CGEs on a single machine or on multiple machines. In such multiple-user scenarios, one or more of each of Controllers, Controller Operators, Service Providers, Eye Trackers, and PMDs could be used.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

The CGE is embodied in one or more executable applications deployable, for example, on desktop or cloud-based computing environments. An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters.

The term GUI, as used herein, may include one or more display images, generated by a display processor and enabling user interaction with a processor or other device and associated data acquisition and processing functions. The GUI may also include an executable procedure or executable application. The executable procedure or executable application conditions the display processor to generate signals representing the GUI display images. These signals are supplied to a display device which displays the image for viewing by the user. The processor, under control of an executable procedure or executable application, manipulates the GUI display images in response to signals received from the input devices. In this way, the user may interact with the display image using the input devices, enabling user interaction with the processor or other device.

The functions and process steps herein may be performed automatically or wholly or partially in response to user command. An activity (including a step) performed automatically is performed in response to one or more executable instructions or device operation without user direct initiation of the activity.

Additionally, in the disclosed technology, therapeutic applications via graphical user interface. The user is not required to wear any sensors or devices. The disclosed technology is designed to be used independently by a large segment of children with ASD without significant involvement of therapists, educators, and parents. In the disclosed technology, therapeutic exercises using GCET are embedded in an immersive 3D video game that captures eye-tracking data (illustrated above) in millisecond intervals relative to the targeted visual behavior of the child. It processes the captured eye-tracking data in near real time to adapt the child's game experience seamlessly and programmatically through the introduction of different forms of guidance and reinforcement, and exercise difficulty level is adjusted based on child performance. In these ways, the disclosed technology intelligently adapts to each child to maintain him or her in a training zone where they are consistently challenged, but not overwhelmed, and enables gradual shaping of targeted behaviors.

Additionally, in this example, the game uses auditory and visual prompts (both stimulus and response), some of which are gaze-contingent, to encourage the player to look at specific emotionally expressive areas of the face which builds on the understanding that individuals with ASD focus on individual facial features when performing ER tasks. In this way, the disclosed technology teaches in a way that closely aligns with current understanding of the different ways in which ER learning occurs in children with ASD. During each session, the platform captures the child's game performance data, including exercise difficulty level attainment and discrete trial success rate, and transmits this data via the internet to a secure cloud-based server where on-demand exercise progress reports can be delivered to therapy and education providers.

In the disclosed technology, the gameplay sessions are comprised of a therapeutic exercise phase and a reward phase that cycle back and forth several times during each session. In the therapeutic exercise phase, the user engages in discrete trials (i.e., repetitions) of gaze-controlled exercises delivered in succession during simulated social interactions with 3D animated cartoon characters. During game exercises, user is prompted to, and receive reinforcement for, focusing their gaze on the eye and mouth regions of game characters and engaging in an emotion matching task. The degree of emotion expression is maintained across images so as to ensure key facial characteristics of each emotion were clearly visible. Gaze pattern data is collected for eye and mouth regions as well as several additional regions of the game character's face using the techniques illustrated above. If the user has difficulty with the exercise task, the game automatically reduces the exercise difficulty level. A token economy is used to provide differential reinforcement; the more accurate the user's exercise performance, the more tokens received, which can later be used to unlock special powers in the reward phase.

In the reward phase, the user enters a virtual video game arcade of different mini-games from which to choose. Following completion of the selected mini-game, the user returns to the therapeutic exercise phase. The frequent cycling of these phases provides a high degree of saliency between exercise performance and reinforcement, minimizes exercise fatigue, and maintains user engagement over time. This enables delivery of an exponential increase in the number of learning opportunities with machine-based consistency and precision.

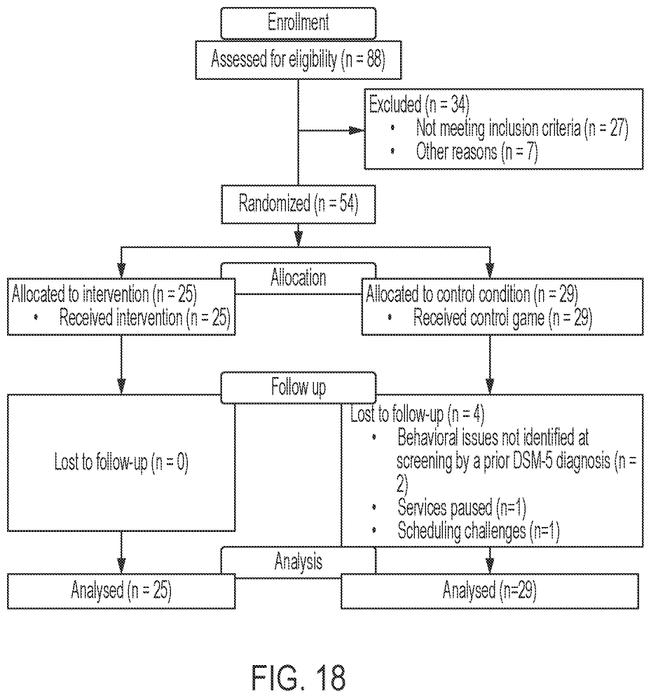

With reference to FIG. 18, the flow diagram illustrates complete participant screening and enrollment information. Families were contacted by phone and screened on initial inclusion criteria (e.g., parent-report of ASD diagnosis, English speaking, prior video game use with a game controller) and exclusion criteria (e.g., sensory-motor difficulties or severe intellectual disability that would preclude use of a computer or mobile computing device). Qualified participants attended an in-person screening to verify additional inclusion criteria; documentation of an ASD diagnosis from a licensed medical professional, and the PPVT-4 and Ekman-60 are conducted to verify inclusion criteria for verbal intelligence and current ER deficits.

After completing screening, children were randomly assigned to either the intervention (n=25) or control (n=29) condition. Participants engaged in video-game play at either their school (n=30), therapy center (n=21) or home (n=3). The individual overseeing participant gameplay (i.e., monitor) received training on how to operate the platform and follow the study protocol. Over approximately six-weeks, participants engage in three to five game sessions per week, with at least 15 minutes of gameplay required for a session to be counted.

In the disclosed technology, children's ER was assessed using the technique as follows. The technique consists of the presentation of 60 faces across 10 actors presenting six basic emotions (i.e., happiness, anger, sadness, fear, surprise, disgust). Facial expressions are displayed on the computer for five seconds, after which the image disappears, and participants select from a list of emotions which best described the facial expression shown. Scores range from 0-60, with higher scores indicating more correct responses. Children completed the procedure in-person at baseline, midpoint (i.e., ˜3 weeks), and post-intervention (i.e., ˜6 weeks); in the event that baseline scores were not available, technique scores from the participant's in-person screening were used as the baseline score. Post-intervention assessments are conducted, on average, two days after the final training session (M=2.26, SD=4.25 days). The disclosed technology sought to examine primary outcomes from baseline to post-therapy, and therefore only data from baseline and post-therapy were used in the current analyses.

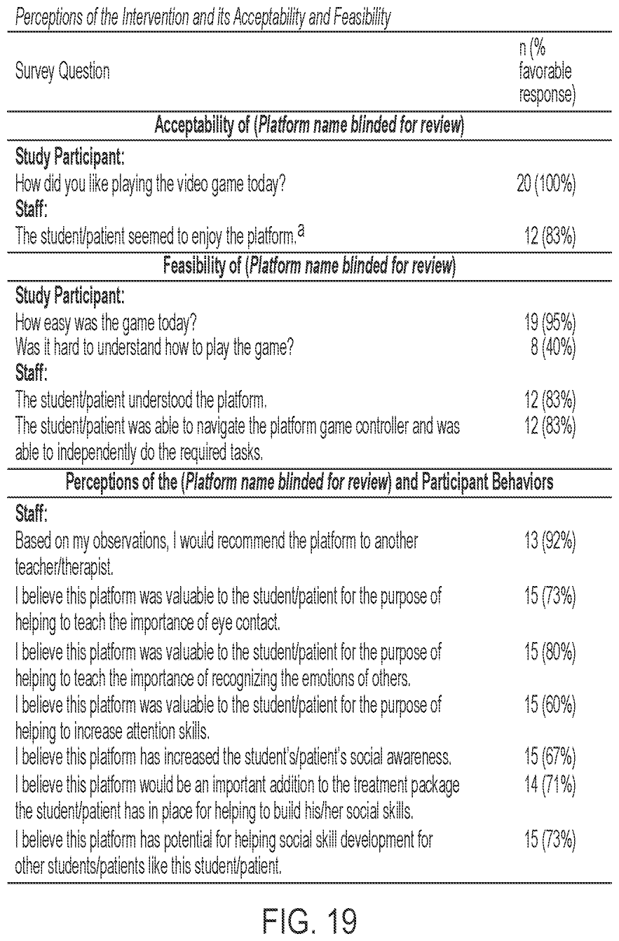

Additionally, a staff member (i.e., one therapist or teacher) at each research site, who had regular interaction with the study participants, completed a survey about feasibility, acceptability, and an overall evaluation of the platform. By way of example, staff rated statements related to acceptability and feasibility of the platform ranging from 0 (“Never true”) to 6 (“Always true”). Furthermore, acceptability was assessed using the statement, “The student/patient seemed to enjoy the disclosed technology.” Feasibility was assessed using the statements, “The student/patient understood the disclosed technology” and “The student/patient was able to navigate the (Platform name blinded) game controller and was able to independently do the required tasks.” Additionally, in this example, staff also rated overall evaluation of the platform and its value in helping teach ER, eye contact, and attention on scales ranging from 0 (“Never”) to 6 (“Always”), 0 (“Not at all valuable”) to 6 (“Extremely valuable”), or 0 (“Not at all”) to 6 (“Very significantly”). Statements used to assess overall evaluation of the platform are presented in the table illustrated in FIG. 19. Rating responses above “Neutral” (e.g., “Sometimes true,” “Usually true,” “Always true”) were combined to determine the percentage of positive responses.

Furthermore, in this example, child perspective of platform feasibility was assessed at the end of the study using the following questions, “Was it hard to understand how to play the game?” (response options: “Yes,” “A little bit,” “No.”) and “How easy was the game today?” (response options, “Hard,” “Medium,” “Easy”). In each case, the latter two response options were combined to assess the percentage of participants where feasibility was demonstrated. Acceptability of the video game was assessed at the end of the study with the question, “How did you like playing the video game today?” Response options were, “I didn't like it,” “I liked it,” and “I liked it a lot.” The latter two responses were combined to determine where acceptability of the game was demonstrated.

Additionally, in this example, data was assessed for normality and outliers, and the intervention and control groups were compared at baseline on demographics, PPVT-4 scores, and the outcome measure to ensure group equivalency. The disclosed technology uses last observation carried forward (LOCF) to impute missing values for the four participants who were lost to follow-up after a baseline Ekman-60 assessment. This imputation method was used given the small amount of missing data and lack of auxiliary data (e.g., IQ, symptom severity) to generate accurate values that account for the missing data pattern in more sophisticated, stochastic imputation methods. A 2 (Time)×2 (Condition) mixed ANOVA was conducted to test for between-group differences in Ekman-60 scores from pre- to post-intervention. If a significant interaction was detected, results were plotted with a bar chart. Cut-offs for interpreting effect sizes (i.e., n2) were based on suggestions provided by Cohen (1988): small=0.01, medium=0.06, large=0.14.

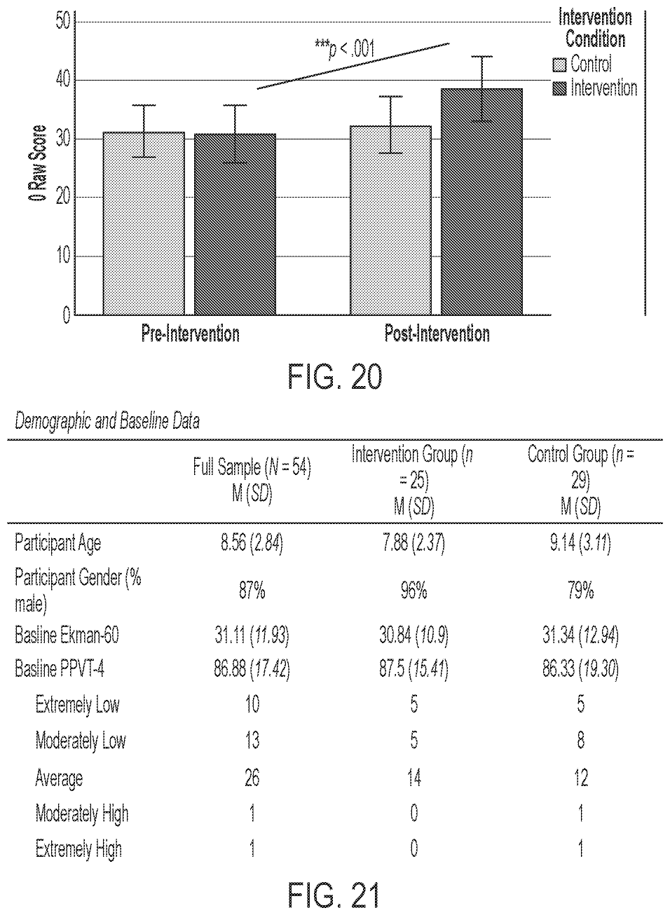

Results from the 2×2 mixed ANOVA revealed a significant Condition×Time interaction, F(1,52)=17.48, p<0.001, partial n²=0.25). As can be seen in FIG. 20, participants in the intervention condition demonstrated significant increases (i.e., ˜25% change) in their Ekman-60 scores from pre- to post-intervention, whereas there was no evidence of change among participants in the control condition. Means, standard deviations, and ranges for Ekman-60 scores by group, across time, can be seen in Table 2. Although qualified by the significant interaction, the disclosed technology detected a main effect of time, F(1,52)=28.38, p<0.001, partial n²=0.35, such that Ekman-60 scores increased from pre- to post-intervention. There was no evidence of a main effect of the intervention condition on Ekman-60 scores, F(1,52)=0.76, p=0.39, partial n2=0.01).

Furthermore, table illustrated in FIG. 21 presents descriptive data for demographics and outcome variables. Participants engaged in an average of 3.12 (SD=0.76) sessions per week and 6.41 (SI)=1.89) total hours of gameplay over the course of the study. The number of sessions per week and total hours of gameplay were similar across conditions (ps>0.05). Children in the intervention group engaged in an average of 577.84 discrete trials throughout the study period (SD=170.5, range 290-892).

In this example, all children in the intervention condition who responded to the acceptability question (n=20) indicated they “Liked’ the game or “Like it a lot.” All children also responded favorably to the feasibility questions, with 60% (n=12) reporting that they understood the game and 95% (n=19) reporting the game was easy (see FIG. 19). Among research site staff's reports on 12 intervention participants, 83% (n=10) indicated that they thought the child enjoyed and understood the intervention and that the child was able to independently perform the required tasks of the intervention. The majority of staff felt that the game was valuable for teaching and/or improving the participants' ER, eye contact, social skills, and attention (see FIG. 19). Nearly all staff (92%) indicated that they would recommend the game to another teacher/therapist.

The disclosed technology provides a large effect for improving objectively measured ER. Additionally, the disclosed technology provides instruction and prompts the learner to attend to faces thereby increasing opportunities to gaze at and respond to faces with different emotions, which research has shown may affect ER skill development. Using GCET, the game delivers immediate conditioned positive reinforcement for gaze directed at key areas of the face for ER (e.g., eyes), areas that individuals with ASD are known to observe less. This contingency may be particularly reinforcing for youth with ASD given high rates of computer and electronic game play in this population and may demonstrate greater effects for achieving desired behavior than rewards and consequences used in many other therapies, such as verbal reinforcement.

The system and processes of the figures are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. As described herein, the various systems, subsystems, agents, managers and processes can be implemented using hardware components, software components, and/or combinations thereof. No claim element herein is to be construed under the provisions of 35 U.S.C. 112(f), unless the element is expressly recited using the phrase “means for.”

The present invention may be a system, a method, and/or a computer program product for managing electronic transactions. The computer program product may include a computer-readable storage medium (or media) having computer-readable program instructions thereon for causing a processor to carry out aspects of the present invention.

Although the disclosed technology has been described with reference to exemplary embodiments, it is not limited thereto. Those skilled in the art will appreciate that numerous changes and modifications may be made to the preferred embodiments of the invention and that such changes and modifications may be made without departing from the true spirit of the disclosed technology. It is therefore intended that the appended claims be construed to cover all such equivalent variations as they fall within the true spirit and scope of the disclosed technology.

What is claimed is:

1. A method comprising:
providing, by a computing device, a three-dimensional gameplay via a graphical user interface to a user device, wherein the provided three-dimensional gameplay prompts a response from a user device and the response is tracked via a gaze contingent technique;
identifying, by the computing device, performance data based on the received response from the user device;
adjusting, by the computing device, a difficulty level of the provided three-dimensional gameplay based on the identified performance data; and
providing, by the computing device, the adjusted three-dimensional gameplay to the user device to track additional performance data.

2. The method as set forth in claim 1 further comprising:
storing, by the computing device, the identified performance data and the additional performance data at a server;
analyzing, by the computing device, the stored performance data and the additional performance data associated with a user of the user device; and
generating, by the computing device, one or more therapeutic progress reports based on the analysis.

3. The method as set forth in claim 1 wherein the provided three-dimensional gameplay comprises simulated social interactions with three-dimensional animated characters.

4. The method as set forth in claim 1 wherein the response comprises data from an eye region, and a mouth region of the user using the user device tracked via the gaze contingent technique.

5. The method as set forth in claim 1 wherein adjusting the three-dimensional gameplay minimizes user fatigue and maintains a user engagement time.

6. The method as set forth in claim 1 wherein the adjusting further comprises, adjusting, by the computing device, the difficulty level of the provided three-dimensional gameplay based on a feedback data received from a therapy or education provider.

7. A non-transitory machine readable medium having stored thereon instructions comprising machine executable code which when executed by at least one machine causes the machine to:
provide a three-dimensional gameplay via a graphical user interface to a user device, wherein the provided three-dimensional gameplay prompts a response from a user device and the response is tracked via a gaze contingent technique;
identify performance data based on the received response from the user device;
adjust a difficulty level of the provided three-dimensional gameplay based on the identified performance data; and
provide the adjusted three-dimensional gameplay to the user device to track additional performance data.

8. The medium as set forth in claim 7 further comprising:
storing the identified performance data and the additional performance data at a server;
analyzing the stored performance data and the additional performance data associated with a user of the user device; and
generating one or more therapeutic progress reports based on the analysis.

9. The medium as set forth in claim 7 wherein the provided three-dimensional gameplay comprises simulated social interactions with three-dimensional animated characters.

10. The medium as set forth in claim 7 wherein the response comprises data from an eye region, and a mouth region of the user using the user device tracked via the gaze contingent technique.

11. The medium as set forth in claim 7 wherein adjusting the three-dimensional gameplay minimizes user fatigue and maintains a user engagement time.

12. The medium as set forth in claim 7 wherein the adjusting further comprises, adjusting, by the computing device, the difficulty level of the provided three-dimensional gameplay based on a feedback data received from a therapy or education provider.

13. A computing device, comprising a memory comprising programmed instructions stored in the memory and one or more processors configured to be capable of executing the programmed instructions stored in the memory to:
provide a three-dimensional gameplay via a graphical user interface to a user device, wherein the provided three-dimensional gameplay prompts a response from a user device and the response is tracked via a gaze contingent technique;
identify performance data based on the received response from the user device;
adjust a difficulty level of the provided three-dimensional gameplay based on the identified performance data; and
provide the adjusted three-dimensional gameplay to the user device to track additional performance data.

14. The device as set forth in claim 13 wherein the one or more processors are further configured to be capable of executing the programmed instructions stored in the memory to:
store the identified performance data and the additional performance data at a server;
analyze the stored performance data and the additional performance data associated with a user of the user device; and
generate one or more therapeutic progress reports based on the analysis.

15. The device as set forth in claim 13 wherein the provided three-dimensional gameplay comprises simulated social interactions with three-dimensional animated characters.

16. The device as set forth in claim 13 wherein the response comprises data from an eye region, and a mouth region of the user using the user device tracked via the gaze contingent technique.

17. The device as set forth in claim 13 wherein adjusting the three-dimensional gameplay minimizes user fatigue and maintains a user engagement time.

18. The device as set forth in claim 13 wherein the adjusting further comprises, adjusting, by the computing device, the difficulty level of the provided three-dimensional gameplay based on a feedback data received from a therapy or education provider.
